(19) United States
(12) Patent Application Publication
(10) Pub. No.: US 2005/0119324 A1
(43) Pub. Date: Jun. 2, 2005

(54) Methods of treating atherosclerosis using NF-kB inhibitors

(75) Inventors: Douglas Carl Harnish, Pennsburg, PA (US); Christopher Cyril Chadwick, West Chester, PA (US)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 10/947,901

(22) Filed: Sep. 23, 2004

Related U.S. Application Data
(60) Provisional application No. 60/506,005, filed on Sep. 24, 2003.

(51) Int. Cl. A61K 31/416

(52) U.S. Cl. 514/406

(57) ABSTRACT

The present invention concerns a method of treating atherosclerosis by diagnosing that a person is in need of treatment for atherosclerosis and administering a therapeutically effective amount of a ligand which modulates NF-kB transcription factor by interaction with estrogen receptor ER-α, estrogen receptor ER-β, or both ER-α and ER-β estrogen receptors with a substantial absence of creatine kinase stimulation. In certain preferred embodiments, the administration is substantially without uterotropic activity.

### CROSS-REFERENCE TO RELATED APPLICATIONS

The present application claims benefit to U.S. Provisional Application Ser. No. 60/506,005, filed Sep. 24, 2003, which is incorporated herein by reference in its entirety.

### FIELD OF THE INVENTION

This invention relates to methods of treatment of atherosclerosis by modulating NFkB transcription with ligands that interact with the estrogen receptor, preferably in the absence of classic estrogenic activity.

### BACKGROUND OF THE INVENTION

The ability of ligands for the estrogen receptor to inhibit inflammatory gene expression causing a reduction of cytokines, chemokines, adhesion molecules and inflammatory enzymes provides a means to treat the inflammatory component of various diseases. Potential therapeutic indications for these types of molecules include type II diabetes (Cefalu, J Womens Health & Gender-based Med. 2001, 10, 241 & Yuan et al., Science, 2001, 293, 1673), osteoarthritis (Pelletier et al. Arthr. & Rheum.,2001, 44:1237 and Felson et al. Curr Opinion Rheum, 1998, 10, 269) asthma (Chin-Chi Lin et. al., Immunol. Lett., 2000, 73, 57), Alzheimer's disease (Roth, A. et. al.; J. Neurosci. Res., 1999, 57, 399) and any other autoimmune based disease. Two types of estrogen receptors have been described (ERα and ERβ) and both are present in most tissues including the intestine (Campbell-Thompson et al., Cancer Research, 2001, 61, 632-640).

A common component of these chronic inflammatory conditions is polymorphonuclear leukocyte and monocyte infiltration into the site of damage through increased expression of cytokines and adhesion molecules responsible for their recruitment. Overproduction of the cytokine interleukin (IL-6) has been associated with states of chronic inflammation (Bauer M. A., Herrmann F., Ann. Hematol. 1991, 62, 203). Synthesis of the IL-6 gene is induced by the transcription factor nuclear factor κB (NF-κB). Interference at this step in the inflammatory process can effectively regulate the uncontrolled proliferative process that occurs in these chronic conditions.

In endothelial cells, 17β-estradiol (E2) inhibits IL-1β induced NF-κB reporter activity and IL-6 expression in an ER dependent fashion (Kurebayashi S., et. al., J. Steroid Biochem. Molec. Biol., 1997, 60, 11). This correlates with anti-inflammatory action of E2 in vivo as confirmed in different animal models of inflammation. In models of atherosclerosis, E2 was shown to protect endothelial cell integrity and function and to reduce leukocyte adhesion and intimal accumulation (Adams, M. R., et al., Arterio., 1990, 1051, Sullivan, T. R. et al. J. Clin. Invst. 1995, 96, 2482, Nathan, L. et. al. '99 Circ. Res., 1999, 85, 377). Similar effects of estrogen on the vascular wall have also been demonstrated in animal models of myocardial infarction (Delyani J A, et al. J. Molec. Cell. Cardiol., 1996, 28, 1001) and congestive heart failure (Feldman '01). Clinically, estrogen replacement therapy (ERT) has been demonstrated to reduce the risk of mortality in patients with both CHF (Reis et. al., J. Am. Coll. Cardio., 2000, 36, 529) and MI (Grodstein, F., et. al., Ann. Int. Med., 2000, 133, 933, Alexander et. al., J. Am. Coll. Cardio., 2001, 38, 1 and Grodstein F. et. al. Ann. Int. Med, 2001, 135, 1). In ERT, clinical studies demonstrated an influence of E2 on the decrease in the production of β-amyloid 1-42 (Aβ42), a peptide central for the formation of senile plaques in Alzheimer's disease (Schonknecht, P. et. al.; Neurosci. Lett., 2001, 307, 122).

Inflammation is recognized as a component of atherosclerosis development, with the transcription factor NF-κB involved in both the early and late stages of the inflammatory-proliferative process. See Turberg et al., Curr. Opin. Lipidol 1998, 9, 387-96. Both activated NF-κB and elevated expression of NF-κB dependent pro-inflammatory gene products including adhesion molecules, cytokines, and chemokines are present in endothelial cells, macrophages and smooth muscle cells with the human atheroma. See Brand et al., J. Clin. Invest. 1996, 97, 1715-22; Bourcier et al., J. Biol. Chem. 1997, 272, 15817; and Reckless et al., Circ. 1999, 99, 2310-16. Treatment of postmenopausal women with estrogens reduces plasma levels of adhesion molecules and other markers of endothelial activation. See Koh et al., Am. J. Cardiol. 1997, 80, 1505 and Ball et al., Fertil. Steril. 1999, 71, 663. In hypercholesterolemic rabbits, estradiol both inhibits monocyte adhesion and decreases BCAM-1 expression in cultured endothelial cells through interference of NF-κB activity. See Simoncini et al., Circ. Res. 2000, 87, 19. These results suggest part of the cardiovascular benefits of estrogen may be due to the ability to interfere with NF-κB mediated inflammatory gene activation in vasculature.

17-β-Estradiol, however, strongly stimulates creatine kinase expression. Thus, in ERT some potential unwanted side effects, such as an increase risk of cardiovascular events in the first year of use, have been demonstrated (Hulley, S. et. al., J. Am. Med. Assoc., 1998, 280, 605) as well as proliferative effects on uterine and breast tissue.

U.S. Pat. Nos. 6,069,175 and 6,124,346 discloses the use of certain estrogen agonist/antagonist compounds in the treatment of atherosclerosis. The compounds are said to inhibit chemokine expression leading to excessive inflammatory cell recruitment.

### SUMMARY OF THE INVENTION

The present invention concerns methods of treating atherosclerosis comprising the steps of identifying a person is need of such treatment for atherosclerosis and administering a therapeutically effective amount of a ligand which modulates NF-κB transcription factor by interaction with estrogen receptor ER-α, estrogen receptor ER-β, or both ER-α and ER-β estrogen receptors, preferably with a substantial absence of creatine kinase stimulation. In certain preferred embodiments, the administration is with a substantial absence of uterotropic activity. Some preferred ligands interact with both ERα and ERβ receptors.

### BRIEF DESCRIPTION OF THE DRAWINGS

FIG. 1 shows that the compound of example 3 provided protection against atherosclerotic lesion development similar to that of EE.

FIG. 2 shows that the beneficial effect on aortic atherosclerosis in the EE-treated group was associated with a significant reduction of serum total cholesterol, VLDL-C and an elevation of HDL-C.

### DETAILED DESCRIPTION OF ILLUSTRATIVE EMBODIMENTS

The present invention provides methods for the treatment of atherosclerosis. Compounds useful in the present invention preferably block interleukin-1β (IL-1β) induced nuclear factor kB (NF-kB) luciferase reporter activity or interleukin-6 (IL-6) expression in an ER dependent fashion in human endothelial cells. Unlike the method disclosed in U.S. Pat. Nos. 6,410,516, 6,150,090, and 5,804,374, the ligands of the instant invention modulate NF-kB activity by interaction with the estrogen receptor rather than direct binding with NF-kB. Particularly preferred ligands are devoid of the proliferative effects on uterine and breast tissue associated with estrogen in vivo. This lack of estrogen side effects is confirmed in vitro by the lack of expression of creatine kinase (CK); a classic estrogen responsive gene. The selective anti-inflammatory compounds described herein are expected to prove useful for the treatment and prevention of chronic inflammatory diseases without stimulating uterine and breast cell proliferation as found with classic estrogens.

One family of compounds useful in the instant invention are substituted 4-(1H-indazol-3-yl)phenols represented by the general formula I and substituted 4-(2H-indazol-3-yl)phenols represented by formula II. Such compounds have been found to be useful for the treatment of the inflammatory component of diseases and particularly in treating atherosclerosis. 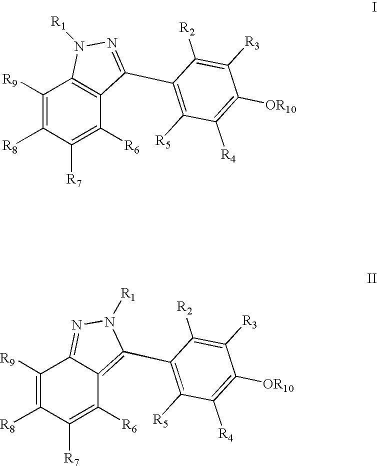
wherein:
R₁ is hydrogen, alkyl, alkenyl, cycloalkyl, cycloalkenyl, aryl, arylalkyl, or a heterocyclic ring system of 4-14 atoms, containing 1-4 heteroatoms selected from N, O, and S;
R₂, R₃, R₄, and R₅, are each, independently, hydrogen, alkyl, alkenyl, hydroxy, alkoxy, aryloxy, halogen, trifluoromethyl, —CN, —NO₂, —CHO, or —CO₂R₁₁;
R₆, R₇, R₈, and R₉, are each, independently, hydrogen, alkyl, alkenyl, hydroxy, alkoxy, aryloxy, halogen, trifluoromethyl, —CO₂R₁₁, aryl, arylalkyl, or a heterocyclic ring system of 4-14 atoms, containing 1-4 heteroatoms selected from N, O, and S;
R₁₀ is hydrogen, —CO₂R₁₁, —CONHR₁₁, —P(═O)(OH)OR₁₁, or —CO(CH₂)ₙCH(NHR₁₂)CO₂R₁₁;
R₁₁ is hydrogen, alkyl, aryl, or arylalkyl;
R₁₂ is hydrogen or —CO₂R₁₁;
n=0-3,
or a pharmaceutically acceptable salt thereof.

In some embodiments,
R₁ is hydrogen, alkyl of 1-6 carbon atoms, alkenyl of 2-7 carbon atoms, cycloalkyl of 3-8 carbon atoms, cycloalkenyl of 4-8 carbon atoms, aryl of 6-20 carbon atoms, arylalkyl of 7-26 carbon atoms, or a heterocyclic ring system of 4-14 atoms, containing 14 heteroatoms selected from N, O, and S;
R₂, R₃, R₄, and R₅, are each, independently, hydrogen, alkyl of 1-6 carbon atoms, alkenyl of 2-7 carbon atoms, hydroxy, alkoxy of 1-6 carbon atoms, aryloxy of 6-20 carbon atoms, halogen, trifluoromethyl, —CN, —NO₂, —CHO, or —CO₂R₁₁;
R₆, R₇, R₈, and R₉, are each, independently, hydrogen, alkyl of 1-6 carbon atoms, alkenyl of 2-7 carbon atoms, hydroxy, alkoxy of 1-6 carbon atoms, aryloxy of 6-20 carbon atoms, halogen, trifluoromethyl, —CO₂R₁₁, aryl of 6-20 carbon atoms, arylalkyl of 7-26 carbon atoms, or a heterocyclic ring system of 4-14 atoms, containing 1-4 heteroatoms selected from N, O, and S; and
R₁₁ is hydrogen, alkyl of 1-6 carbon atoms, aryl of 6-20 carbon atoms, or arylalkyl of 7-26 carbon atoms.

The compounds of formula I and formula II can be converted to salts, in particular pharmaceutically acceptable salts using art recognized procedures. The compounds of formulas I and II that have a basic center can form acid addition salts. These are formed, for example, with strong inorganic acids, such as mineral acids for example sulfuric acid, phosphoric acid or a hydrohalic acid, with strong organic carboxylic acids, such as alkanecarboxylic acids of 1 to 4 carbon atoms which are unsubstituted or substituted, for example, by halogen, for example acetic acid such as saturated or unsaturated dicarboxylic acids, for example oxalic, malonic, succinic, maleic, fumaric, phthalic, or terephthalic acid, such as hydroxycarboxylic acids, for example ascorbic, glycolic, lactic, malic, tartaric or citric acid, such as amino acids, for example aspartic or glutamic acid, or such as benzoic acid, or with organic sulfonic acids, such as alkane- (of 1 to 4 carbon atoms) or arylsulfonic acids, for example methane- or p-toluenesulfonic acid. Corresponding acid addition salts can also be formed having, if desired, an additionally present basic center. The compounds of formula I having at least one acid group can form salts with bases. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Salts which are unsuitable for pharmaceutical uses but which can be employed, for example, for the isolation or purification of free compounds I or their pharmaceutically acceptable salts, are also included.

Some preferred 4-(1-H-indazol-3-yl)phenols useful in this invention include those of Group A in which:
R₁ is alkyl of 1-6 carbon atoms, alkenyl of 2-7 carbon atoms, cycloalkyl of 3-8 carbon atoms, cycloalkenyl of 4-8 carbon atoms, or a heterocyclic ring system of 4-14 atoms, containing 1-4 heteroatoms selected from N, O, and S;
R₂ is hydrogen, alkyl of 1-6 carbon atoms, alkenyl of 2-7 carbon atoms, hydroxy, alkoxy of 1-6 carbon atoms, or halogen;
R₇ and R₉, are each, independently, hydrogen, alkyl of 1-6 carbon atoms, hydroxy, halogen, trifluoromethyl, —CO₂R₁₁, aryl of 6-20 carbon atoms, arylalkyl of 7-26 carbon atoms, or a heterocyclic ring system of 4-14 atoms, containing 1-4 heteroatoms selected from N, O, and S,
where the remaining substituents are as defined above.

Other preferred compounds of this invention include those of group B in which:
R₁ is alkyl of 1-6 carbon atoms, alkenyl of 2-7 carbon atoms, cycloalkyl of 3-8 carbon atoms, or cycloalkenyl of 4-8 carbon atoms;
R₂ is hydrogen, alkyl of 1-6 carbon atoms, halogen, or hydroxy;
R₉ is alkyl of 1-6 carbon atoms, halogen, trifluoromethyl, —CO₂R₁₁, aryl of 6-20 carbon atoms, arylalkyl of 7-26 carbon atoms, or a heterocyclic ring system of 4-14 atoms, containing 1-4 heteroatoms selected from N, O, and S;
R₁₀ is hydrogen;
where the remaining substituents are as defined above.

Yet other preferred compounds of this invention include those of C in which:
R₁ is alkyl of 1-6 carbon atoms or alkenyl of 2-7 carbon atoms;
R₉ is alkyl of 1-6 carbon atoms, halogen, or trifluoromethyl, where the remaining substituents are as defined above.

The reagents used in the preparation of the compounds of this invention can be either commercially obtained or can be prepared by standard procedures described in the literature. Compounds of formula I and formula II wherein R₁₀═H can be prepared from a common precursor of formula III as outlined in Scheme 1. 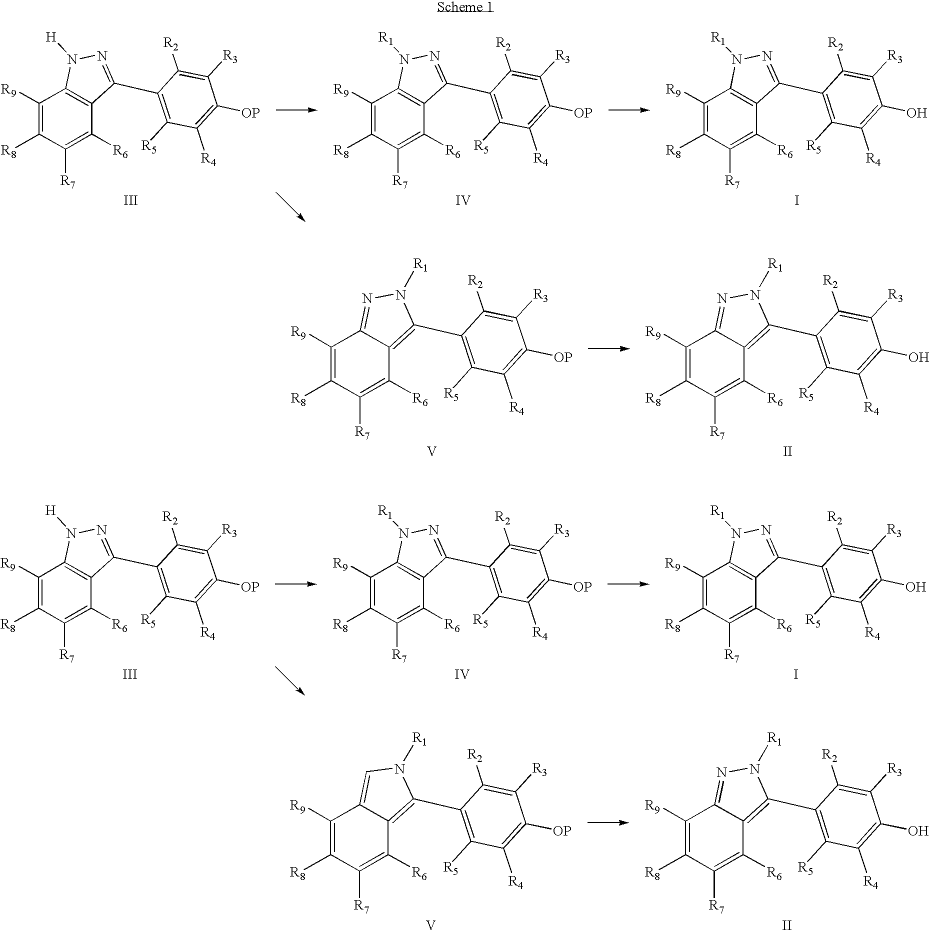
where
R₁ is alkyl, cycloalkyl, alkenyl, cycloalkenyl, alkyloxy;
R₂, R₃, R₄, R₅, R₆, R₇, R₈, and R₉ are as previously defined; and
P is a phenol protecting group, preferably but not limited to methyl, benzyl or t-butyldiphenylsilyl.

Thus, compounds of formula III preferably are treated with sodium hydride in a suitable solvent such as 4-dimethylaminopyridine (DMAP). When the gas evolution ceases, the alkyl halide is added and the solution is heated at 50° C. overnight. The reaction is partitioned with ethyl acetate and water. The organic phase is dried with a suitable drying agent such as sodium sulfate (Na₂SO₄). The crude products IV and V are isolated as a single residue after filtration and concentration of the organic layer in vacuo. Separation is easily carried out by chromatography known to one skilled in the art, to provide the separated intermediates IV and V.

Compounds of formula I and formula II preferably are prepared from IV or V respectively by a deprotection step.

When P is benzyl, deprotection to the phenol preferably is accomplished by hydrogenation over 10% palladium on carbon using either hydrogen gas, or catalytic hydride transfer with cyclohexene or ammonium formate.

When P is methyl, deprotection preferably is carried out using BBr₃ with cyclohexene as a scavenger for HBr.

When P is t-butyldiphenylsilyl, deprotection can be accomplished with tetrabutylammonium fluoride.

Compounds of formula V can also be prepared as outlined in Scheme 2 from compounds of formula VI.

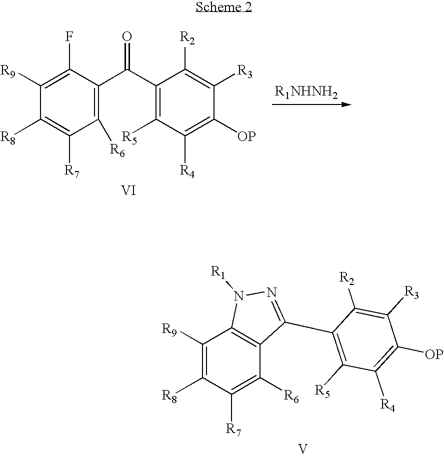

2-Fluorobenzophenones of compound VI can be reacted directly with optimally substituted hydrazines where R₁ is alkyl or aryl, which are either commercially available or readily prepared by common procedures known to those skilled in the art. Thus, a mixture of the benzophenones of compound VI are combined with the hydrazines in a suitable solvent such as methanol in the presence of ethyl acetate. The intermediate hydrazone either spontaneously cyclizes to the compounds of formula IV or can be isolated by concentration of the reaction mixture. The isolated hydrazone is heated neat to temperatures of up to 190° C. The residues are purified by chromatography to provide compounds of formula IV.

Compounds of formula I, wherein R₂ and R₈ are OH and R₃, R₄, R₅, R₆, R₇, and R₉ are hydrogen, can also be prepared by a similar process from commercially available 2,2′,4,4′-tetrahydrobenzophenone according to the literature preparation of R. Krishnan, S. A. Lang, Y. I. Lin, R. G. Wilkinson J. Heterocycl. Chem, 1988, 25, 447 and outlined in Scheme 3.

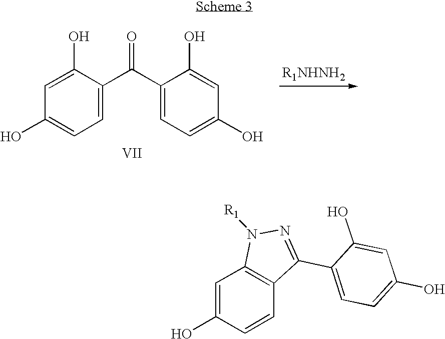

Thus, a solution of the substituted hydrazine salt (1 to 2 equivalents), sodium acetate (1 to 4 equivalents) and 2,2′,4,4′-tetrahydroxybenzophenone (1 equivalent) in an appropriate solvent such as methanol (0.2 molar solution) is stirred at ambient temperature overnight. The reaction mixture is concentrated in vacuo and the residues partitioned with EtOAc and H₂O. The organic phase is dried (Na₂SO₄) and concentrated in vacuo to give the intermediate hydrazone. The residues are heated at 190° C. overnight. Product residues are purified by chromatography.

Compounds of formula III can be readily prepared from compounds of formula VI as shown in Scheme 4.

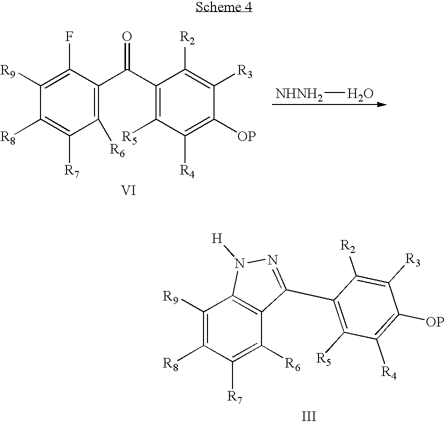

Thus, an appropriately substituted compound of formula VI is reacted with an excess of hydrazine hydrate in pyridine containing DMAP. The reaction is heated at 100° C. for at least 24 hours. The reaction is concentrated in vacuo and the residue is partitioned with ethyl acetate and 1 N HCl. The organic phase is washed with brine and dried with a drying agent such as Na₂SO₄. The solvent is evaporated to provide the compounds of formula III.

Compounds of formula VI can be readily prepared as outlined in Scheme 5 from the reaction of an appropriately substituted 2-fluoro-N-methoxy-N-methyl-benzamide of formula VII. 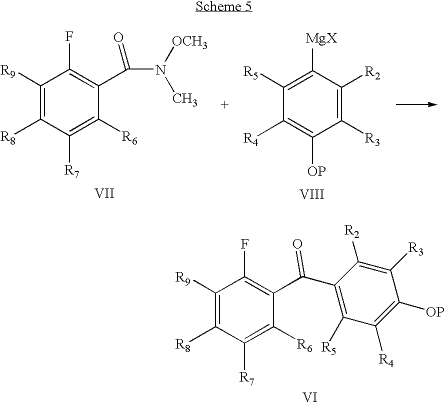
where X is preferably but not limited to Br. Compounds of formula VIII are either commercially available or readily prepared by one skilled in the art. One suitable solvent is tetrahydrofuran (THF).

The Weinreb amides of formula VII can are generated by the reaction of an appropriately substituted 2-fluorobenzoic acid with N,O-dimethylhydroxylamine and N,N-carbonyldiimidazole in a suitable solvent such as DMF (Robertson et. al., J. Med. Chem., 1990, 33, 3167) or from the acid chloride prepared from reaction of the benzoic acid with oxalyl chloride in a suitable solvent such as THF in the presence of a base such as N,N-diisopropylethylamine.

Compounds of formula IV can also be prepared as outlined in Scheme 6 Scheme 6 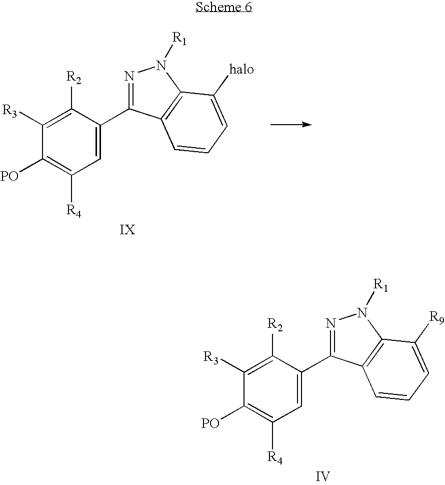
where
R₁, R₂, R₃, and R₄ are as defined above;
and halo is Cl or Br.

Thus, when halo is Br, compounds of formula IV where R₉ is aryl, heteroaryl, heterocycle, and alkenyl, can be prepared by the Suzuki coupling of IX with an appropriately substituted boronic acid in a suitable solvent such as dioxane, in the presence of an aqueous base such as potassium carbonate, in the presence of 1 to 5 mol % of palladium catalyst such as tetrakis(triphenylphoshine)palladium (0). The mixture is typically heated at 80° C. for a period of 1 to 24 hours (see Miyaura, N. Suzuki, A., Chem Rev., 1995, 95, 2457). The compounds are obtained in pure forms by chromatography known to those skilled in the art.

When halo is Cl, compounds of formula IV where R₉ is aryl, heteroaryl, heterocyclic can be prepared as described by Huang J. and Nolan S. P., et al, J. Am. Chem Soc., 1999, 121, 9889. Thus, reaction of IX with a suitably substituted aryl magnesium bromide in a suitable solvent such as dioxane in the presence of an N-heterocyclic carbene ligand and a palladium catalyst such as but not limited to palladium(II)acetate.

Compounds of formula V can be prepared as outlined in Scheme 7. 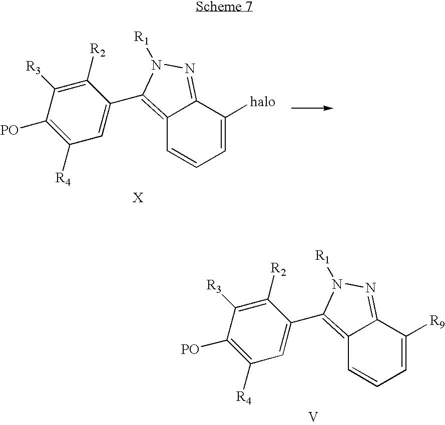
where R₁, R₂, R₃, and R₄ are as defined above; and halo is Cl or Br. Thus, compounds of formula V where R₉ is aryl, heteroaryl, heterocyclic, and alkenyl, can be prepared in an analogous fashion to the regioisomer described above in Scheme 6.

Prodrugs of formula I and formula II can readily be prepared as described below.

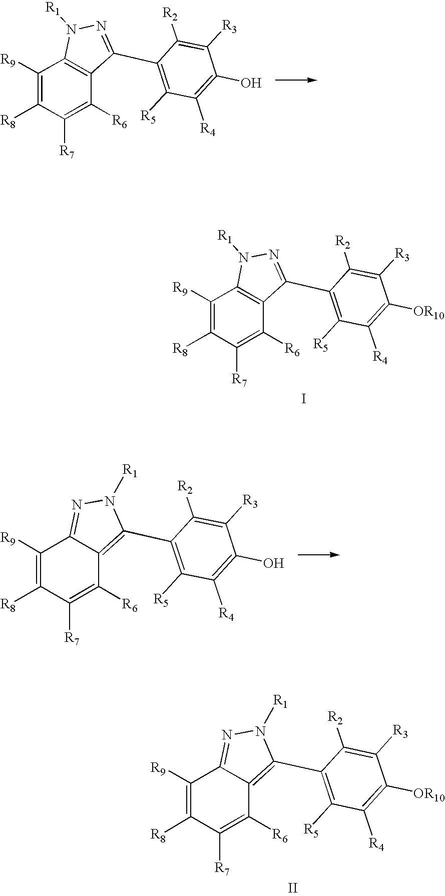

Thus when R₁₀ is COOR₁₁, compounds can be prepared by methods commonly known to those skilled in the art. The reaction of an acid chloride with compounds of formula I and formula II wherein R₁ is H in a suitable solvent such as methylene chloride in the presence of a suitable base such as N,N-diisopropylethylamine affords the ester prodrugs.

For amino acid esters, standard coupling techniques known to those skilled in the art can be used, including activation of the carboxylic acid in the presence of DMAP (Boden E. P., Keck, G. E., J. Org. Chem, 1985, 50, 2394). A solution of compounds of formulas I and II dicyclohexylcarbodiimide and DMAP in a suitable solvent such as CH₂Cl₂ is stirred overnight at ambient temperature. The reaction mixture is purified typically by column chromatography known to those skilled in the art to provide the ester.

When R₁₀ is CONHR₁₁ , compounds of formula I and II may be reacted with substituted isocyanates in a suitable solvent such as dioxane and heated at 80° C. for up to 48 hours. (March's Adv. Org. Chem, 5^{th} ed, 16: 1183, Wiley Interscience, 2001).

When R₁₀ is P(═O)(OH)OR₁₁, the substituted hydrogen phosphates of compounds of formulas I and II can be prepared as described by Rodriguez, M. J. et al., Bioorg. Med. Chem. Lett., 1999, 9, 1863. Thus, a solution of compounds of formulas I or II, wherein R₁₀ is H substituted dichlorophosphate and lithium hexamethyldisilazide in a suitable solvent such as THF is stirred for 1 hour at ambient temperature. The reaction mixture is quenched with H₂O and purified by reversed phase HPLC, known by one skilled in the art.

Other useful compounds of the invention are dihydrophenanthridinesulfonamide compounds of formulae (XI) or (XII): 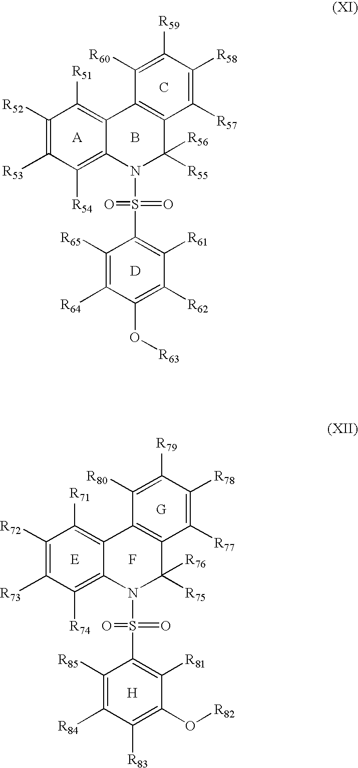
wherein
R₅₁, R₅₂, R₅₃, R₅₄, R₅₇, R₅₈, R₅₉, R₆₀, R₆₁, R₆₂, R₆₄, and R₆₅ are each, independently, hydrogen, R₆₇, monofluoroalkyl, monofluoroalkenyl, aryl-R₆₆—, heteroaryl-R₆₆—, hydroxyalkyl, HO—R₆₆—, R₆₇—X—R₆₆—, HS—R₆₆—, R₆₇—S(O)—, R₆₇—S(O)₂—, R₆₇—SO₃—, R₆₇—S(O)₂NR′—, —N(R′)₂, —NR′—C(NH₂)═NR′, cyano, nitro, halogen, —OR′, —SR′, —SO₃R′, —S(O)₂N(R′)₂, —C(O)R′, —C(R′)═N—OR′, —C(NH₂)═NR′, —CO₂R′, —OC(O)R′, or —C(O)N(R′)₂; or are linked with either Rₚ₊₁ or Rₚ₋₁ by an -alkylene-, or —X-alkylene- group;
R₅₅ is hydrogen, R₆₇, monofluoroalkyl, monofluoroalkenyl, aryl-R₆₆—, heteroaryl-R₆₆—, hydroxyalkyl, HO—R₆₆—, R₆₇—X—R₆₆—, HS—R₆₆—, —C(O)R′, —CO₂R′, or —C(O)N(R′)₂; or R₅₅ may be linked with either R₅₆ or R₅₇ and linked with an -alkylene- or —X-alkylene- group;
R₅₆ is hydrogen, R₆₇, monofluoroalkyl, monofluoroalkenyl, aryl-R₆₆—, heteroaryl-R₆₆—, hydroxyalkyl, HO—R₆₆—, R₆₇—X—R₆₆—, HS—R₆₆—, —C(O)R′, —CO₂R′, or —C(O)N(R′)₂; or R₆₆ may be linked with either R₅₅ or R₅₇ and linked with an -alkylene- or —X-alkylene- group;
R₆₃ is R′, R₆₇—X—R₆₆—, R₆₇—S(O)—, R₆₇—S(O)₂—, —SO₃R′, —S(O)₂N(R′)₂, or D-glucuronidate;
R₆₆ is -alkylene-, -cycloalkylene-, -alkylene-X-alkylene-, -alkylene-X-cycloalkylene-, -cycloalkylene-X-alkylene-, or -cycloalkylene-X-cycloalkylene-;
R₆₇ is alkyl, aryl, heteroaryl, cycloalkyl, alkenyl, cycloalkenyl, alkynyl, alkenyl-X-alkylene-, cycloalkenyl-X-alkylene-, or perfluoroalkyl;
R′ is, independently, hydrogen, alkyl, alkenyl, alkynyl, cycloalkyl, cycloalkenyl, monofluoroalkyl, perfluoroalkyl, aryl, arylalkyl, heteroaryl, heteroarylalkyl, hydroxy-(C₂-C₆)alkyl, alkoxyalkyl, alkylthioalkyl, formyl, acyl, alkoxycarbonyl, —C(O)NH₂, alkylaminocarbonyl, dialkylaminocarbonyl, alkylaminoalkyl, or dialkylaminoalkyl; or when an atom contains two R′ groups, the R′ groups may be linked with an -alkylene- group;
X is O, —NR′—, —S(O)ₘ—, —C(O)—, —OC(O)—, —C(O)O—, —NR′C(O)—, or —C(O)NR′—;
m is 0, 1, or 2;
p is 52, 53, 56, 57, 58, 59, 62, 63, or 64;
R₇₁, R₇₂, R₇₃, R₇₄, R₇₇, R₇₈, R₇₉, R₈₀, R₈₁, R₈₃, R₈₄, and R₈₅ are, independently, hydrogen, R₆₇, monofluoroalkyl, monofluoroalkenyl, aryl-R₆₆—, heteroaryl-R₆₆—, hydroxyalkyl, HO—R₆₆—, R₆₇—Y—R₆₆—, HS—R₆₆—, R₆₇—S(O)—, R₆₇—S(O)₂—, R₆₇—SO₃—, R₆₇—S(O)₂NR′—, —N(R′)₂, —NR′—C(NH₂)═NR′, cyano, nitro, halogen, —OR′, —SR′, —SO₃R′, —S(O)₂N(R′)₂, —C(O)R′, —C(R′)═N—OR′, —C(NH₂)═NR′, —CO₂R′, —OC(O)R′, or —C(O)N(R′)₂; or are linked with either R_{q+1} or R_{q-1} by an -alkylene-, or —Y-alkylene- group;
R₇₅ is hydrogen, R₆₇, monofluoroalkyl, monofluoroalkenyl, aryl-R₆₆—, heteroaryl-R₆₆—, hydroxyalkyl, HO—R₆₆—, R₆₇—Y—R₆₆—, HS—R₆₆—, —C(O)R′, —CO₂R′, or —C(O)N(R′)₂; or R₂₅ may be linked with either R₇₆ or R₇₇ by an -alkylene- or -Y-alkylene- group;
R₇₆ is hydrogen, R₆₇, monofluoroalkyl, monofluoroalkenyl, aryl-R₆₆—, heteroaryl-R₆₆—, hydroxyalkyl, HO—R₆₆—, R₆₇—Y—R₆₆—, HS—R₆₆—, —C(O)R′, —CO₂R′, or —C(O)N(R′)₂; or R₇₆ may be linked with either R₂₅ or R₂₇ by an -alkylene- or -Y-alkylene- group;
R₈₂ is R′, R₆₇—Y—R₆₆—, R₆₇—S(O)—, R₆₇—S(O)₂—, —SO₃R′, —S(O)₂N(R′)₂, or D-glucuronidate;
Y is O, —NR′—, —S(O)ₙ—, —C(O)—, —OC(O)—, —C(O)O—, —NR′C(O)—, or —C(O)NR′—;
n is 0, 1, or 2;
q is 72, 73, 76, 77, 78, 79, 82, 83, or 84;
or pharmaceutically acceptable salts thereof.

Preferred dihydrophenanthridinesulfonamides compounds useful in the invention include those of the Groups A-F detailed below.

Group A compounds are those of formula (XII), where the remaining substituents are as defined above.

Group B compounds include those of group A where R_{32′} is hydrogen and the remaining substituents are as defined above.

The compounds of group C include those of group B in which:
R₇₁, R₇₂, R₇₃, R₇₄, R₇₇, R₇₈, R₇₉, R₈₀, R₈₁, R₈₃, R₈₄, and R₈₅ are each independently, hydrogen, R₆₇, aryl-R₆₆—R₆₇—Y—R₆₆—, hydroxyalkyl, HO—R₆₆-, halogen, —OR′, —COR′, or —CO₂R′;
R₇₅ and R₇₆ are each, independently, hydrogen or R₆₇;
R₆₆ is -alkylene-;
R₆₇ is alkyl, aryl, heteroaryl, or perfluoroalkyl;
R′ is hydrogen or alkyl; and
where the remaining substituents are as defined above.

Group D compounds include those in which the compound is of formula (I) and the remaining substituents are as defined above.

The compounds of group E include the compounds of group D where R₆₃ is hydrogen and the remaining substituents are as defined above.

Group F compounds include those of group E in which:
R₅₁, R₅₂, R₅₃, R₅₄, R₅₇, R₅₈, R₅₉, R₆₀, R₆₁, R₆₂, R₆₄, and R₆₅ are each independently, hydrogen, R₆₇, aryl-R₆₆—, R₆₇—X—R₆₆—, hydroxyalkyl, HO—R₆₆—, halogen, —OR′, —COR′, or —CO₂R′;
R₅₅, and R₅₆, are each, independently, hydrogen or R₆₇;
R₆₆ is -alkylene-;
R₆₇ is alkyl, aryl, heteroaryl, or perfluoroalkyl;
R′ is hydrogen or alkyl;
where the remaining substituents are as defined above.

Yet other compounds useful in the invention are of the formula: 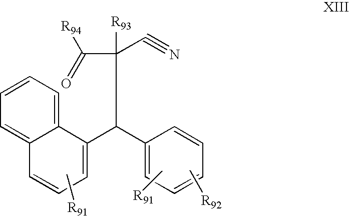
wherein
R₉₁ and R₉₂ are each independently hydrogen, halo, alkyl, alkoxy, nitro, cyano, thioalkyl, CF₃, OCF₃, or hydroxy;
R₉₃ is H, alkyl, allyl, benzyl, alkenyl, cycloalkyl methyl, or heteroaryl methyl;
R₉₄ is NR₉₅R₉₆, morpholinyl, thiomorpholinyl, t-butylamino, 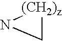
   where z is an integer from 2 to 7 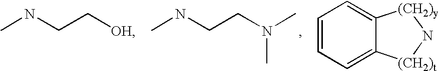
   where y is an integer from 1 to 3, and
t is 0 or 1, 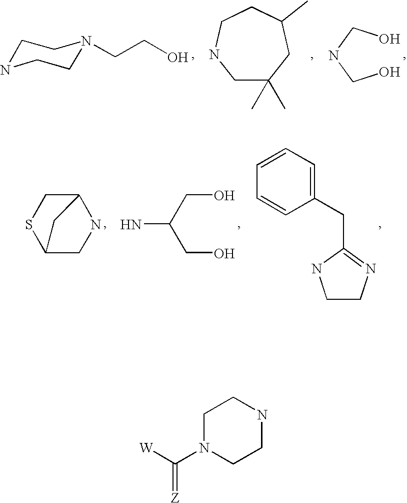
   where Z is O or S; and
W is O-aryl, O-heteroaryl, NH-aryl, or NH-heteroaryl;
4-benzylpiperazinyl, 4-heteroarylmethyl piperazinyl, 4-arylmethyl piperazinyl, heteroarylpiperazine, arylpiperazine, heteroaryl tetrahydropyridine, aryl tetrahydropyridine, heteroarylpiperidine, arylpiperidine, or OR₉₆;
R₉₅ and R₉₆ are each, independently, alkyl, heteroaryl methyl, aryl methyl, or cycloalkyl;
   and pharmaceutically acceptable salts, hydrates and solvates thereof.

In certain embodiments,
R₉₁ and R₉₂ are each independently hydrogen, halo, alkyl of 1 to 6 carbon atoms, alkoxy of 1 to 6 carbon atoms, nitro, cyano, thioalkyl, CF₃, OCF₃, or hydroxy;
R₉₃ is H, alkyl of 1 to 6 carbon atoms, allyl, benzyl, alkenyl of 2 to 7 carbon atoms, cycloalkyl methyl, or heteroaryl methyl;
R₉₄ is NR₉₅R₉₆, morpholinyl, thiomorpholinyl, t-butylamino, 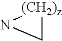
   where z is an integer from 2 to 7 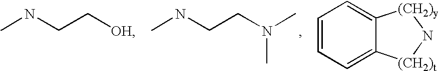
   where y is an integer from 1 to 3, and
t is 0 or 1, 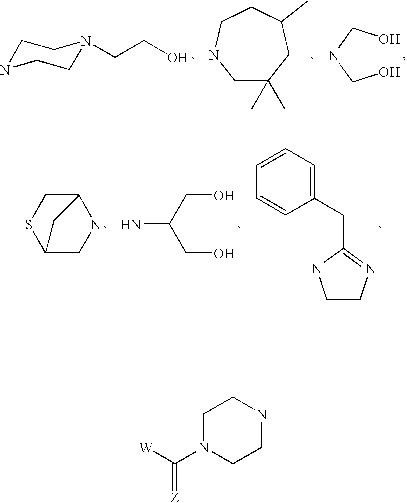
   where Z is O or S; and
W is O-aryl, O-heteroaryl, NH-aryl, or NH-heteroaryl;
4-benzylpiperazinyl, 4-heteroarylmethyl piperazinyl, 4-arylmethyl piperazinyl, heteroarylpiperazine, arylpiperazine, heteroaryl tetrahydropyridine, aryl tetrahydropyridine, heteroarylpiperidine, arylpiperidine, or OR₉₆; and
R₉₅ and R₉₆ are each, independently, alkyl of 1 to 6 carbon atoms, heteroaryl methyl, aryl methyl, or cycloalkyl of 3 to 8 carbon atoms.

Another class of cyanopropanoic acid derivatives is described as 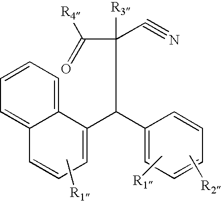
wherein
R₉₁ and R₉₂ are each independently hydrogen, halo, alkyl, alkoxy, nitro, cyano, thioalkyl, CF₃, OCF₃, or hydroxy;
R₉₃ is H, alkyl, allyl, benzyl, or alkenyl, cycloalkyl methyl, or heteroaryl methyl;
R₉₄ is NR₉₅R₉₆, t-butylamino, 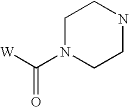
   where W is O-heteroaryl, O-aryl, NH-aryl, or NH-heteroaryl,
4-benzyl piperazinyl, 4-heteroarylmethyl piperazinyl, 4-arylmethyl piperazinyl, heteroarylpiperazine, heteroarylpiperazine, heteroaryl tetrahydropyridine, aryl tetrahydropyridine, heteroarylpiperidine, arylpiperidine, OR₉₆; and
R₉₅ and R₉₆ are each independently alkyl, benzyl, alkylene, heteroaryl methyl, aryl methyl, or cycloalkyl.

Another preferred class of cyanopropanoic acid derivatives is described as 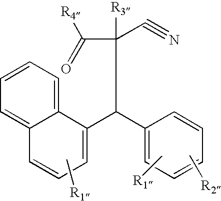
R₉₁ and R₉₂ are independently hydrogen, halo, alkyl, alkoxy, CF₃, OCF₃, or hydroxy;
R₉₃ is H, alkyl, allyl, benzyl, alkenyl, or aryl methyl, or heteroaryl methyl;
R₉₄ is NR₉₅R₉₆, 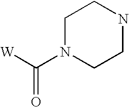
   where W is O-aryl,
4-benzyl piperazinyl, 4-heteroarylmethyl piperazinyl, 4-arylmethyl piperazinyl, heteroarylpiperazine, arylpiperazine, heteroaryl tetrahydropyridine, aryl tetrahydropyridine, heteroarylpiperidine, arylpiperidine, or OR₉₆; and
R₉₅ and R₉₆ are each independently alkyl, benzyl, heteroaryl methyl, aryl methyl or cycloalkyl.
   Definitions

The term “alkyl”, employed alone, is defined herein as, unless otherwise stated, either a (C₁-C₂₀) straight chain or (C₃-C₂₀) branched-chain monovalent saturated hydrocarbon moiety. Examples of saturated hydrocarbon alkyl moieties include, but are not limited to, chemical groups such as methyl, ethyl, n-propyl, isopropyl, n-butyl, tert-butyl, isobutyl, sec-butyl; higher homologs such as n-pentyl, n-hexyl, n-heptyl, n-octyl, and the like. It is preferred that straight chain alkyl moieties have 1-6 carbon atoms, and branched alkyl moieties have 3-8 carbon atoms.

The term “alkenyl”, employed alone, is defined herein as, unless otherwise stated, either a (C₂-C₂₀) straight chain or (C₃-C₂₀) branched-chain monovalent hydrocarbon moiety containing at least one double bond. Such hydrocarbon alkenyl moieties may be mono or polyunsaturated, and may exist in the E or Z configurations. The compounds of this invention are meant to include all possible E and Z configurations. Examples of mono or polyunsaturated hydrocarbon alkenyl moieties include, but are not limited to, chemical groups such as vinyl, 2-propenyl, isopropenyl, crotyl, 2-isopentenyl, butadienyl, 2-(butadienyl), 2,4-pentadienyl, 3-(1,4-pentadienyl), and higher homologs, isomers, and the like. It is preferred that straight chain alkenyl moieties have 2-7 carbon atoms, and branched alkenyl moieties have 3-8 carbon atoms.

The term “alkynyl”, employed alone, is defined herein as, unless otherwise stated, either a (C₂-C₂₀) straight chain or (C₃-C₂₀) branched-chain monovalent hydrocarbon moiety containing at least one triple bond. Examples of alkynyl moieties include, but are not limited to, chemical groups such as ethynyl, 1-propynyl, 1-(2-propynyl), 3-butynyl, and higher homologs, isomers, and the like. It is preferred that straight chain alkynyl moieties have 2-7 carbon atoms, and branched alkynyl moieties have 3-8 carbon atoms.

The term “alkylene”, employed alone or in combination with other terms, is defined herein as, unless otherwise stated, either a (C₁-C₂₀) straight chain or (C₂-C₂₀) branched-chain bivalent hydrocarbon moiety derived from an alkane; or a (C₂-C₂₀) straight chain or branched-chain bivalent hydrocarbon moiety derived from an alkene. Such hydrocarbon alkylene moieties may be fully saturated, or mono or polyunsaturated, and may exist in the E or Z configurations. The compounds of this invention are meant to include all possible E and Z configurations. Examples of saturated and unsaturated hydrocarbon alkylene moieties include, but are not limited to, bivalent chemical groups such as —CH₂—, —CH₂CH₂—, —CH₂CH₂CH₂—, —CH₂CH₂CH₂CH₂—, —CH═CH—, —CH═CHCH═CH—, vinylidene, and higher homologs, isomers, and the like. Preferred alkylene chains have 2-7 carbon atoms.

The term “cycloalkyl”, employed alone or in combination with other terms, is defined herein as, unless otherwise stated, a monocyclic, bicyclic, tricyclic, fused, bridged, or spiro monovalent saturated hydrocarbon moiety of 3-10 carbon atoms, wherein the carbon atoms are located inside or outside of the ring system. In some embodiments, the ring comprises 3-8 carbon atoms. Any suitable ring position of the cycloalkyl moiety may be covalently linked to the defined chemical structure. Examples of cycloalkyl moieties include, but are not limited to, chemical groups such as cyclopropyl, cyclopropylmethyl, cyclobutyl, cyclopentyl, cyclohexyl, cyclohexylmethyl, cyclohexylethyl, cycloheptyl, norbornyl, adamantyl, spiro[4.5]decanyl, and homologs, isomers, and the like.

The term “cycloalkenyl”, employed alone or in combination with other terms, is defined herein as, unless otherwise stated, a monocyclic, bicyclic, tricyclic, fused, bridged, or spiro monovalent unsaturated hydrocarbon moiety of 3-10 carbon atoms containing at least one double bond, wherein the carbon atoms are located inside or outside of the ring system. In some embodiments, the ring has 4-8 carbon atoms. Any suitable ring position of the cycloalkenyl moiety may be covalently linked to the defined chemical structure. Examples of cycloalkenyl moieties include, but are not limited to, chemical groups such as cyclopropenyl, cyclopropenylmethyl cyclobutenyl, cyclopentenyl, cyclohexenyl, cyclohexenylmethyl, cyclohexenylethyl, cycloheptenyl, norbornenyl, and homologs, isomers, and the like.

The term “cycloalkylene”, employed alone, is defined herein as, unless otherwise stated, a bivalent moiety of 3-10 carbon atoms derived from a monocyclic, bicyclic, tricyclic, fused, bridged, or spiro hydrocarbon. In some embodiments, the ring has 4-8 carbon atoms. Such hydrocarbon cycloalkylene moieties may be fully saturated, or mono or polyunsaturated, and may exist in the E or Z configurations. The compounds of this invention are meant to include all possible E and Z configurations. Any suitable ring position of the cycloalkylene moiety may be covalently linked to the defined chemical structure. Examples of saturated and unsaturated hydrocarbon cycloalkylene moieties include, but are not limited to, bivalent chemical groups such as cyclopropylene, cyclopentylene, cyclohexylene, cyclohexenylene, trans-decahydronaphthalenylene, spiro[3.3]heptenylene, and higher homologs, isomers, and the like.

The terms “halo” or “halogen”, employed alone or in combination with other terms, is defined herein as, unless otherwise stated, a fluorine, chlorine, bromine, or iodine atom.

The term “monofluoroalkyl”, employed alone, is defined herein as, unless otherwise stated, either a (C₁-C₁₀) straight chain or (C₃-C₁₀) branched-chain monovalent saturated hydrocarbon moiety containing only one fluorine atom. Examples of monofluoroalkyl moieties include, but are not limited to, chemical groups such as —CH₂F, —CH₂CH₂F, —CH(CH₃)CH₂CH₂F, and higher homologs, isomers, and the like. Preferred chain lengths are from 1-6 carbon atoms for straight chains and from 3-8 carbon atoms for branched chains.

The term “monofluoroalkenyl”, employed alone, is defined herein as, unless otherwise stated, either a (C₂-C₁₀) straight chain or (C₃-C₁₀) branched-chain monovalent unsaturated hydrocarbon moiety, containing only one fluorine atom and at least one double bond. Examples of monofluoroalkenyl moieties include, but are not limited to, chemical groups such as —CH═CH₂F, —CH₂CH═CH₂F, —CH═CHCH₂F, —C(CH₃)═CHF and higher homologs, isomers, and the like. Preferred chain lengths are from 2-7 carbon atoms for straight chains and from 3-8 carbon atoms for branched chains.

The term “perfluoroalkyl”, employed alone or in combination with other terms, is defined herein as, unless otherwise stated, either a (C₁-C₁₀) straight chain or (C₃-C₁₀) branched-chain monovalent saturated hydrocarbon moiety containing two or more fluorine atoms. Examples of perfluoroalkyl moieties include, but are not limited to, chemical groups such as trifluoromethyl, —CH₂CF₃, —CF₂CF₃, and —CH(CF₃)₂, and homologs, isomers, and the like. Preferred chain lengths are from 1-7 carbon atoms for straight chains and from 3-8 carbon atoms for branched chains.

The term “aryl”, employed alone or in combination with other terms, is defined herein as, unless otherwise stated, an aromatic carbocyclic moiety of up to 20 carbon atoms, which may be a single ring (monocyclic) or multiple rings (bicyclic, up to three rings) fused together or linked covalently. Any suitable ring position of the aryl moiety may be covalently linked to the defined chemical structure. Examples of aryl moieties include, but are not limited to, chemical groups such as phenyl, 1-naphthyl, 2-naphthyl, dihydronaphthyl, tetrahydronaphthyl, biphenyl, anthryl, phenanthryl, fluorenyl, indanyl, biphenylenyl, acenaphthenyl, acenaphthylenyl, and the like. It is preferred that the aryl moiety contain 6-14 carbon atoms.

The term “arylalkyl”, employed alone or in combination with other terms, is defined herein as, unless otherwise stated, an aryl group, as herein before defined, suitably substituted on any open ring position with an alkyl moiety wherein the alkyl chain is either a (C₁-C₆) straight or (C₂-C₇) branched-chain saturated hydrocarbon moiety. In certain embodiments, arylalkyl groups have 7 to 26 carbon atoms. Examples of arylalkyl moieties include, but are not limited to, chemical groups such as benzyl, 1-phenylethyl, 2-phenylethyl, diphenylmethyl, 3-phenylpropyl, 2-phenylpropyl, fluorenylmethyl, and homologs, isomers, and the like.

The term “heterocyclic ring system” is defined as being 4 to 14 carbon atoms. he rings may contain from one to four hetero atoms selected from nitrogen (N), oxygen (O), or sulfur (S), wherein the nitrogen or sulfur atom(s) are optionally oxidized, or the nitrogen atom(s) are optionally substituted or quarternized. Any suitable ring position of the heteroaryl moiety may be covalently linked to the defined chemical structure. The ring may be saturated, unsaturated, or partially unsaturated. Heterocyclic rings may comprise a single ring or a multiple ring system comprising up to three rings.

The term “heteroaryl”, employed alone or in combination with other terms, is defined herein as, unless otherwise stated, an aromatic heterocyclic ring system, which may be a single ring (monocyclic) or multiple rings (bicyclic, up to three rings) fused together or linked covalently. The rings may contain from one to four hetero atoms selected from nitrogen (N), oxygen (O), or sulfur (S), wherein the nitrogen or sulfur atom(s) are optionally oxidized, or the nitrogen atom(s) are optionally substituted or quarternized. Any suitable ring position of the heteroaryl moiety may be covalently linked to the defined chemical structure. Examples of heteroaryl moieties include, but are not limited to, heterocycles such as furan, thiophene, pyrrole, N-methylpyrrole, pyrazole, N-methylpyrazole, imidazole, N-methylimidazole, oxazole, isoxazole, thiazole, isothiazole, 1H-tetrazole, 1-methyltetrazole, 1,3,4-oxadiazole, 1H-1,2,4-triazole, 1-methyl-1,2,4-triazole, 1,3,4-triazole, 1-methyl-1,3,4-triazole, pyridine, pyrimidine, pyrazine, pyridazine, benzoxazole, benzisoxazole, benzothiazole, benzofuran, benzothiophene, thianthrene, dibenzo[b,d]furan, dibenzo[b,d]thiophene, benzimidazole, N-methylbenzimidazole, indole, indazole, quinoline, isoquinoline, quinazoline, quinoxaline, purine, pteridine, 9H-carbazole, β-carboline, and the like.

The term “heteroarylalkyl”, employed alone or in combination with other terms, is defined herein as, unless otherwise stated, a heteroaryl group, as herein before defined, suitably substituted on any open ring position with an alkyl moiety, wherein the alkyl chain is either a (C₁-C₆) straight or (C₂-C₇) branched-chain saturated hydrocarbon moiety. Examples of heteroarylalkyl moieties include, but are not limited to, chemical groups such as furanylmethyl, thienylethyl, indolylmethyl, and the like.

Heteroaryl chemical groups, as herein before defined, also include saturated or partially saturated heterocyclic rings. Examples of saturated or partially saturated heteroaryl moieties include, but are not limited to, chemical groups such as azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, tetrahydroisoquinolinyl, and the like.

The term “acyl”, employed alone or in combination with other terms, is defined herein as, unless otherwise stated, either an alkyl, arylalkyl, heteroarylalkyl, (C₂-C₁₀) straight chain, or (C₄-C₁₁) branched-chain monovalent hydrocarbon moiety; wherein the carbon atom, covalently linked to the defined chemical structure, is oxidized to the carbonyl oxidation state. Such hydrocarbon moieties may be mono or polyunsaturated, and may exist in the E or Z configurations. The compounds of this invention are meant to include all possible E and Z configurations. Examples of acyl moieties include, but are not limited to, chemical groups such as acetyl, propionyl, butyryl, 3,3-dimethylbutyryl, trifluoroacetyl, pivaloyl, hexanoyl, hexenoyl, decanoyl, benzoyl, nicotinyl, isonicotinyl, and homologs, isomers, and the like.

The term “hydroxyalkyl”, employed alone or in combination with other terms, is defined herein as, unless otherwise stated, a (C₁-C₁₀) straight chain hydrocarbon, terminally substituted with a hydroxyl group. Examples of hydroxyalkyl moieties include chemical groups such as —CH₂OH, —CH₂CH₂OH, —CH₂CH₂CH₂OH, and higher homologs.

The term “alkoxy”, employed alone or in combination with other terms, is defined herein as, unless otherwise stated, either a (C₁-C₁₀) straight chain or (C₃-C₁₀) branched-chain hydrocarbon covalently bonded to an oxygen atom. Examples of alkoxy moieties include, but are not limited to, chemical groups such as methoxy, ethoxy, isopropoxy, sec-butoxy, tert-butoxy, decanoxy, and homologs, isomers, and the like. In certain embodiments, the alkoxy group has 1-6 carbon atoms.

The terms “aryloxy” or “heteroaryloxy”, employed alone or in combination with other terms, or unless otherwise stated, are aryl or heteroaryl groups, as herein before defined, which are further covalently bonded to an oxygen atom. Examples of aryloxy, or heteroaryloxy moieties include, but are not limited to, chemical groups such as C₆H₅O—, 4-pyridyl-O—, and homologs, isomers, and the like.

The term “carbonyl”, employed alone or in combination with other terms, is defined herein as, unless otherwise stated, a bivalent one-carbon moiety further bonded to an oxygen atom with a double bond. An example is

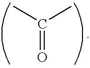

The term “alkoxycarbonyl”, employed alone or in combination with other terms, is defined herein as, unless otherwise stated, an alkoxy group, as herein before defined, which is further bonded to a carbonyl group to form an ester moiety. Examples of alkoxycarbonyl moieties include, but are not limited to, chemical groups such as methoxycarbonyl, ethoxycarbonyl, isopropoxycarbonyl, sec-butoxycarbonyl, tert-butoxycarbonyl, decanoxycarbonyl, and homologs, isomers, and the like.

The term “alkylthio”, employed alone or in combination with other terms, is defined herein as, unless otherwise stated, either a (C₁-C₁₀) straight chain or (C₃-C₁₀) branched-chain hydrocarbon moiety covalently bonded to a sulfur atom. Examples of alkylthio moieties include, but are not limited to, chemical groups such as methylthio, ethylthio, isopropylthio, sec-butylthio, tert-butylthio, decanylthio, and homologs, isomers, and the like. It is preferred that straight chain alkylthio moieties have 1-6 carbon atoms, and branched alkylthio moieties have 3-8 carbon atoms.

The terms “arylthio” or “heteroarylthio”, employed alone or in combination with other terms, or unless otherwise stated, are aryl or heteroaryl groups, as herein before defined, which are further covalently bonded to a sulfur atom. Examples of arylthio or heteroarylthio moieties include, but are not limited to, chemical groups such as C₆H₅S—, 4-pyridyl-S—, and homologs, isomers, and the like.

The terms “alkoxyalkyl” or “alkylthioalkyl”, employed alone or in combination with other terms, are an alkoxy or alkylthio group, as herein before defined, which is further covalently bonded to an unsubstituted (C₁-C₁₀) straight chain or unsubstituted (C₂-C₁₀) branched-chain hydrocarbon. Examples of alkoxyalkyl or alkylthioalkyl moieties include, but are not limited to, chemical groups such as, methoxymethyl, methylthioethyl, ethylthioethyl, isopropylthiomethyl, sec-butylthioethyl, —CH₂CH(CH₃)OCH₂CH₃, and homologs, isomers, and the like. It is preferred that straight chain alkoxyalkyl or alkylthioalkyl moieties have 1-6 carbon atoms, and branched alkoxyalkyl or alkylthioalkyl moieties have 3-8 carbon atoms.

The terms “aryloxyalkyl”, “heteroaryloxyalkyl”, “arylthioalkyl”, or “heteroarylthioalkyl”, employed alone or in combination with other terms, or unless otherwise stated, are aryloxy, heteroaryloxy, arylthio, or heteroarylthio groups, as herein before defined, which are further covalently bonded to an unsubstituted (C₁-C₁₀) straight chain or unsubstituted (C₂-C₁₀) branched-chain hydrocarbon. Examples of aryloxyalkyl, heteroaryloxyalkyl, arylthioalkyl, or heteroarylthioalkyl moieties include, but are not limited to, chemical groups such as C₆H₅OCH₂—, C₆H₅OCH(CH₃)—, 4-pyridyl-O—CH₂CH₂—, C₆H₅SCH₂—, C₆H₅SCH(CH₃)—, 4-pyridyl-S—CH₂CH₂—, and homologs, isomers, and the like. It is preferred that straight chain aryloxyalkyl, heteroaryloxyalkyl, arylthioalkyl, or heteroarylthioalkyl moieties have 1-6 carbon atoms, and branched aryloxyalkyl, heteroaryloxyalkyl, arylthioalkyl, or heteroarylthioalkyl moieties have 3-8 carbon atoms.

The term “alkylamino”, employed alone or in combination with other terms, or unless otherwise stated, is a moiety with one alkyl group, wherein the alkyl group is an unsubstituted (C₁-C₆) straight chain hereunto before defined alkyl group or an unsubstituted (C₃-C₈) hereunto before defined cycloalkyl group. Examples of alkylamino moieties include, but are not limited to, chemical groups such as —NH(CH₃), —NH(CH₂CH₃), —NH-cyclopentyl, and homologs, and the like.

The term “dialkylamino”, employed alone or in combination with other terms, or unless otherwise stated, is a moiety with two independent alkyl groups, wherein the alkyl groups are unsubstituted (C₁-C₆) straight chain hereunto before defined alkyl groups or unsubstituted (C₃-C₈) hereunto before defined cycloalkyl groups. Two groups may be linked to form an unsubstituted (C₁-C₆) -alkylene- group. Examples of dialkylamino moieties include, but are not limited to, chemical groups such as —N(CH₃)₂, —N(CH₂CH₃)₂, —NCH₃(CH₂CH₃), 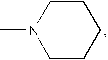
and homologs, and the like.

The term “alkylaminoalkyl” employed alone or in combination with other terms, or unless otherwise stated, is an alkylamino moiety, as herein before defined, which is further covalently bonded to a straight chain alkyl group of 1-6 carbon atoms. Examples of alkylaminoalkyl moieties include, but are not limited to, chemical groups such as —CH₂NH(CH₃), —CH₂CH₂NH(CH₂CH₃), —CH₂CH₂CH₂NH(CH₂CH₃), and homologs, and the like.

The term “dialkylaminoalkyl” employed alone or in combination with other terms, or unless otherwise stated, is a dialkylamino moiety, as herein before defined, which is further covalently bonded to a straight chain alkyl group of 1-6 carbon atoms. Examples of dialkylaminoalkyl moieties include, but are not limited to, chemical groups such as —CH₂N(CH₃)₂, —CH₂CH₂N(CH₂CH₃)₂, —CH₂CH₂CH₂NCH₃(CH₂CH₃), and homologs, and the like.

The terms “alkylaminocarbonyl” or “dialkylaminocarbonyl”, employed alone, or unless otherwise stated, are alkylamino or dialkylamino moieties, as herein before defined, which are further bonded to a carbonyl group. Examples of alkylaminocarbonyl or dialkylaminocarbonyl moieties include, but are not limited to, chemical groups such as —C(O)NH(CH₃), —C(O)N(CH₂CH₃)₂, —C(O)NCH₃(CH₂CH₃), and homologs, and the like.

Each of the above terms (e.g., alkyl, aryl, heteroaryl) includes unsubstituted, monosubstituted, and polysubstituted forms of the indicated radical or moiety. Representative substituents for each type of moiety are provided below.

Substituents for alkyl, alkenyl, alkynyl, cycloalkyl, cycloalkenyl, alkylene, cycloalkylene, the alkyl portion of arylalkyl and heteroarylalkyl, saturated or partially saturated heterocyclic rings, and acyl or carbonyl moieties can be, employed alone or in combination with other terms, —R′, OR′, ═O, ═NR′, ═N—OR′, —NR′R″, —SR′, halo, trifluoromethyl, trifluoromethoxy, —OC(O)R′, —CO₂R′, —C(O)NR′R″, —OC(O)NR′R″, —NR″C(O)R′, —NR″CO₂R′, —NR′C(O)NR′R″, —NH—C(NH₂)═NH, —NR′C(NH₂)═NH, —NH—C(NH₂)═NR′, —S(O)R′, —S(O)₂R′, —S(O)₂NR′R″, cyano, and nitro; wherein, R′ or R″ are each, independently, hydrogen, unsubstituted (C₁-C₆)alkyl, unsubstituted (C₃-C₇)cycloalkyl, aryl, aryl-(C₁-C₃)alkyl, aryloxy-(C₁-C₃)alkyl, arylthio-(C₁-C₃)alkyl, heteroaryl, heteroaryl-(C₁-C₃)alkyl, heteroaryloxy-(C₁-C₃)alkyl, or heteroarylthio-(C₁-C₃)alkyl groups; or if optionally taken together may be linked as an -alkylene- group to form a ring.

The aryl or heteroaryl moieties, employed alone or in combination with other terms, may be optionally mono-, di- or tri-substituted with substituents selected from the group consisting of —R′, —OR′, —SR′, —C(O)R′, —CO₂R′, —alkoxyalkyl, alkoxyalkyloxy, cyano, halogen, nitro, trifluoromethyl, trifluoromethoxy, —NR′R″; alkylaminoalkyl, dialkylaminoalkyl, hydroxyalkyl, —S(O)R′, —S(O)₂R′, —SO₃R′, —S(O)₂NR′R″, —CO₂R′, —C(O)NR′R″, —OC(O)NR′R″, —NR″C(O)R′, —NR″CO₂R′, —NR′C(O)NR′R″, —NH—C(NH₂)═NH, —NR′C(NH₂)═NH, —NH—C(NH₂)═NR′, —S(O)R′, and —S(O)₂R′; wherein, R′ or R″ are each, independently, hydrogen, (C₁-C₆)alkyl, (C₃-C₇)cycloalkyl, aryl, aryl-(C₁-C₃)alkyl, aryloxy-(C₁-C₃)alkyl, arylthio-(C₁-C₃)alkyl, heteroaryl, heteroaryl-(C₁-C₃)alkyl, heteroaryloxy-(C₁-C₃)alkyl, or heteroarylthio-(C₁-C₃)alkyl groups; or if optionally taken together may be linked as an -alkylene- group to form a ring.

As used herein, the term “a substantial absence of creatine kinease stimulation” means the compound has an IC₅₀ value greater than 1 μmol with an efficacy of less than 30% compared to 17-β-estradiol.

The phrase “a substantial absence of uterotropic activity” means that no statistically significant uterine wet weight gain is observed.

A pro-drug is defined as a compound which is convertible by in vivo enzymatic or non-enzymatic metabolism (e.g. hydrolysis) to a compound of the invention.

The compounds of the present invention may contain an asymmetric atom, and some of the compounds may contain one or more asymmetric atoms or centers, which may thus give rise to optical isomers (enantiomers) and diastereomers. While shown without respect to the stereochemistry in Formula (I) or (II), the present invention includes such optical isomers (enantiomers) and diastereomers (geometric isomers); as well as the racemic and resolved, enantiomerically pure R and S stereoisomers; as well as other mixtures of the R and S stereoisomers and pharmaceutically acceptable salts thereof. Optical isomers may be obtained in pure form by standard procedures known to those skilled in the art, and include, but are not limited to, diasteromeric salt formation, kinetic resolution, and asymmetric synthesis. It is also understood that this invention encompasses all possible regioisomers, and mixtures thereof, which may be obtained in pure form by standard separation procedures known to those skilled in the art, and include, but are not limited to, column chromatography, thin-layer chromatography, and high-performance liquid chromatography.

The compounds of the present invention may contain isotopes of atoms for diagnostic, therapeutic, or metabolic purposes. Such isotopes may or may not be radioactive.

The compounds of this invention include racemates, enantiomers, geometric isomers, or pro-drugs of the compounds described herein.

As used herein, the term “pharmaceutically acceptable” refers to a carrier medium which does not interfere with the effectiveness of the biological activity of the active ingredient(s) and which is not toxic to the host to which it is administered.

Pharmaceutically acceptable salts of the compounds of the invention with an acidic moiety can be formed from organic and inorganic bases. Suitable salts with bases are, for example, metal salts, such as alkali metal or alkaline earth metal salts, for example sodium, potassium, or magnesium salts; or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Similarly, when a compound of the present invention contains a basic moiety, salts can be formed from organic and inorganic acids. For example salts can be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known pharmaceutically acceptable acids.

As used in accordance with this invention, the term “providing,” with respect to providing a compound or substance covered by this invention, means either directly administering such a compound or substance, or administering a pro-drug, derivative, or analog which will form the effective amount of the compound or substance within the body. This invention also covers providing the compounds of this invention to treat the disease states disclosed herein that the compounds are useful for treating.

As used herein, the terms “therapeutically effective amount” and “therapeutically effective dose” as applied to the active ingredient refers to the amount of the component in the composition or administered to the host that results in an increase in the therapeutic index of the host. The “therapeutic index” can be defined for purposes herein in terms of efficacy, i.e., extent of reduction or inhibition of inflammation. Suitable doses of the active ingredient can be determined using well-known methods, a variety of which are known and readily available in the pharmaceutical sciences, including, for example, measurement of markers associated with the disorder, the biological effects of TNF-α, and decreased symptomatology.

It is understood that the effective dosage of the active compounds of this invention may vary depending upon the particular compound utilized, the mode of administration, the condition, and severity thereof, of the condition being treated, as well as the various physical factors related to the individual being treated. It is projected that compounds of this invention will be administered at an oral daily dosage of from about 0.05 mg to about 30 mg per kilogram of body weight, preferably administered in divided doses two to six times per day, or in a sustained release form. For most large mammals, the total daily dosage is from about 3.5 mg to about 2100 mg, preferably from about 3.5 to about 5 mg. In the case of a 70 kg human adult, the total daily dose will generally be from about 3.5 mg to about 2100 mg and may be adjusted to provide the optimal therapeutic result.

The compounds of this invention can be formulated neat or with a pharmaceutical carrier for administration, the proportion of which is determined by the solubility and chemical nature of the compound, chosen route of administration and standard pharmacological practice. The pharmaceutical carrier may be solid or liquid.

A solid carrier can include one or more substances which may also act as flavoring agents, sweetening agents, lubricants, solubilizers, suspending agents, fillers, glidants, compression aids, binders, or tablet-disintegrating agents; it can also be an encapsulating material. In powders, the carrier is a finely divided solid which is in admixture with the finely divided active ingredient.

Solid dosage unit forms or compositions such as tablets, troches, pills, capsules, powders, and the like, may contain a solid carrier binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose, or saccharin. When a dosage unit form is a capsule, it may contain, in addition to materials of the above type, a liquid carrier such as a fatty oil. Various other materials may be present as coatings or to modify the physical form of the dosage unit. For instance, tablets may be coated with shellac, sugar or both.

Liquid carriers are used in preparing liquid dosage forms such as solutions, suspensions, dispersions, emulsions, syrups, elixirs and pressurized compositions. The active ingredient can be dissolved or suspended in a pharmaceutically acceptable liquid carrier such as water, an organic solvent, a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, preferably sodium carboxymethyl cellulose solution); alcohols, including monohydric alcohols such as ethanol and polyhydric alcohols such as glycols and their derivatives; lethicins, and oils such as fractionated coconut oil and arachis oil. For parenteral administration, the liquid carrier can also be an oily ester such as ethyl oleate and isopropyl myristate. Sterile liquid carriers are useful in sterile liquid form compositions for parenteral administration. The liquid carrier for pressurized compositions can be a halogenated hydrocarbon or other pharmaceutically acceptable propellant.

A liquid pharmaceutical composition such as a syrup or elixir may contain, in addition to one or more liquid carriers and the active ingredients, a sweetening agent such as sucrose, preservatives such as methyl and propyl parabens, a pharmaceutically acceptable dye or coloring agent, or a flavoring agent such as cherry or orange flavoring.

Liquid pharmaceutical compositions which are sterile solutions or suspensions can be administered intraocularly or parenterally, for example, by intramuscular, intraperitoneal or subcutaneous injection. Sterile solutions can also be administered intravenously. The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy injectability exists. It must be stable under the conditions of manufacture and storage and must be preserved against the contaminating action of microorganisms such as bacteria and fungi. The carrier can be a solvent or dispersion medium containing a liquid carrier, for example, water, ethanol, polyol (e.g. glycerol, propylene glycol and liquid polyethylene glycol), suitable mixtures thereof, and vegetable oils. The liquid carrier may be suitably mixed with a surfactant such as hydroxypropylcellulose.

The compounds of the present invention may also be administered rectally or vaginally in the form of a conventional suppository. For administration by intranasal or intrabronchial inhalation or insufflation, the compounds of this invention may be formulated into an aqueous or partially aqueous solution, which can then be utilized in the form of an aerosol. The compounds of this invention may be administered topically, or also transdermally through the use of a transdermal patch containing the active compound and a carrier that is inert to the active compound, which is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semipermeable membrane covering a reservoir containing the active ingredient with or without a carrier, or a matrix containing the active ingredient. Other occlusive devices are known in the literature.

### EXAMPLES

The following describes the preparation of representative compounds of this invention. Compounds described as homogeneous were determined to be 98% or greater a single peak (exclusive of enantiomers) by analytical reverse phase chromatographic analysis with 254 nM UV detection. Melting points are reported as uncorrected in degrees centigrade. The infrared data is reported as wave numbers at maximum absorption, vₘₐₓ in reciprocal centimeters, cm⁻¹. Mass spectral data is reported as the mass-to-charge ratio, m/z; and for high resolution mass spectral data, the calculated and experimentally found masses, [M+H]⁺, for the neutral formulae M are reported. Nuclear magnetic resonance data is reported as δ in parts per million (ppm) downfield from the standard, tetramethylsilane; along with the solvent, nucleus, and field strength parameters. The spin-spin homonuclear coupling constants are reported as J values in hertz; and the multiplicities are reported as a: s, singlet; d, doublet; t, triplet; q, quartet; quintet; or br, broadened. Italicized elements or groups are those responsible for the chemical shifts. The yields given below are for informational purposes and may vary according to experimental conditions or individual techniques.

Representative compounds of this invention were evaluated in the following standard pharmacological test procedures which demonstrated their anti-inflammatory activity. The test procedures used and the results obtained are briefly described below.

### Example 1

### 4-(1,7-disubstituted-1H-indazol-3-yl)phenols

Step A: A solution of (2-fluoro-3-substituted-phenyl)(4-methoxy-2-substituted-phenyl)methanone (1 equivalent), hydrazine hydrate (10 eq.) and DMAP (1 eq.) in pyridine was heated at 100° C. for 24-48 hrs. The cooled reaction mixture was partitioned with EtOAc and 1 N HCl. The organic phase was washed with brine and dried (Na₂SO₄). The resulting residue was purified by flash chromatography to give the intermediate 3-(4-methoxyphenyl)-7-substituted-1-1H-indazole.

Step B: A solution of the intermediate 3-(4-methoxyphenyl)-7-substituted-1-1H-indazole (1 eq.) in DMF was added in one portion sodium hydride (1 eq., 60% in oil). After the gas evolution ceased, the alkyl halide was added and the reaction was stirred at ambient to 50° C. overnight. The cool reaction mixture was partitioned with EtOAc and 1 N HCl. The organic phase was washed with brine and dried (Na₂SO₄). The resulting residue was purified by flash chromatography or by HPLC chromatography through silica gel columns 150×12 mm (Biotage) at 10 mL/min with methyl-t-butyl ether/hexane (gradient elution 1:9 to 1:1) to give the intermediates 3-(4-methoxyphenyl)-7-substituted-1-substituted-1H-indazole and 3-(4-methoxyphenyl)-7-substituted-2-substituted-2H-indazole.

Step C: A solution of 3-(4-methoxyphenyl)-7-substituted-(1 or 2-substituted)-(1H or 2H)-indazole (1 eq.) in CH₂Cl₂ containing excess equivalents of cyclohexene at −78° C. was treated with boron tribromide (4 eq.) and slowly allowed to warm to ambient temperature. The reaction was quenched by dropwise edition of CH₃OH to the cooled reaction. The solvent was removed in vacuo and the residue partitioned with EtOAc and 1 N HCl. The organic phase was washed with brine and dried (Na₂SO₄). Removal of the solvent in vacuo afforded the crude product. Pure product was obtained by crystallization or flash chromatography through water deactivated silica gel.

Note: HPLC retention times were obtained using the following conditions:

| | |
|---|---|
| Column: | Keystone Aquasil C18 (50 × 2 mm, 5 u), |
| Solvent System: | A: 95% 10 mM NH4OAc/5% acetonitrile, B: 95% |
| | acetonitrile 5% 10 mM NH₄OAc, |
| Gradient | 0% B to 100% B over 0-15 minutes, |
| Flow | 0.8 mL/min |
| Detection: | UV. various wavelengths |

### Example 2

### 4-[1-allyl-7-(trifluoromethyl)-1H-indazol-3-yl]benzene-1,3-diol

### Step 1: 1-allyl-3-(2,4-dimethoxyphenyl)-7-(trifluoromethyl)-1H-indazole

Prepared according to Example 1, step B from 3-(2,4-methoxyphenyl)-7-trifluoromethyl-1H-indazole 0.52 g, 1.6 mmol), sodium hydride (60% in oil, 0.065 g, 1.6 mmol) and allyl bromide (0.138 mL, 1.6 mmol) to give the title compound (0.26 g) as a white solid.

¹H NMR (DMSO-d₆): δ 3.73 (s, 3H), 3.80 (s, 3H), 4.85 (dd, 1H, J=1.5 and 14.65), 5.1 (m, 3H), 5.97-6.05 (m, 1H), 6.39 (dd, 1H, J=2.32 and 6.14), 6.64 (s, 1H), 7.25 (t, 1H), 7.35 (d, 1H), 7.85-7.87 (m, 2H),).

MS (ESI) m/z 363 [M+H]+.

### Step 2: 4-[1-allyl-7-(trifluoromethyl)-1H-indazol-3-yl]benzene-1,3-diol

Prepared according to Example 1, step C from l-allyl-3-(2,4-dimethoxyphenyl)-7-(trifluoromethyl)-1H-indazole (0.065 g, 0.18 mmol), boron tribromide (0.136 mL, 1.4 mmol) and 1.0 mL of cyclohexene to give the product (0.066 g) as a white solid,

mp 114-115° C.;

¹H NMR (DMSO-d₆): δ 4.87 (dd, 1H, J=1.37 and 17.10 Hz), 5.31-5.08 (m, 3H), 6.01-6.08 (m, H), 6.39 (dd, 1H, J=2.44 and 8.40 Hz), 6.46 (s, 1H), 7.30 (t, 1H), 3.78 (d, 1H), 7.85-7.87 (m, 1H), 8.14-8.19 (m, 1H), 9.59 (broad s, 1H), 9.82 (broad s, 1H)

MS (ESI) m/z 335 [M+H]+.

Anal. calcd for C₁₇H₁₃F₃N₂O₂: C, 61.08; H, 3.92; N, 8.38; Found: C, 61.02; H, 3.76; N, 8.28

### Example 3

### 4-[(8-Fluoro-6-methylphenanthridin-5(6H)-yl)sulfonyl]phenol

### Step 1: N-(4′-Fluorobiphenyl-2-yl)acetamide

A stirred solution of 2-iodoaniline (32.6 g, 149 mmol) and 4-fluorophenylboronic acid (20.8 g, 149 mmol) in tetrahydrofuran (1.5 L) was treated under nitrogen with [1,1′-bis(diphenylphosphino)ferrocene]dichloropalladium (II) complex with dichloromethane (2.20 g, 2.69 mmol) and a 5 N sodium hydroxide solution (60 mL). The reaction mixture was heated at reflux for twelve hours, cooled to room temperature, and the solvent removed in vacuo. The residue was dissolved in ethyl acetate (250 mL) and extracted with a saturated, aqueous, sodium chloride solution (100 mL). The aqueous phase was further extracted with ethyl acetate (2×50 mL). The combined organic phase was dried over anhydrous magnesium sulfate, filtered, and concentrated in vacuo to a brown oil. The brown oil was filtered through a short column of silica gel, and eluted with a mixture of ethyl acetate-hexane (1:4). After evaporation of the solvent in vacuo, a solution of the crude 4′-fluoro-biphenyl-2-ylamine in dichloromethane (75 mL) was treated with pyridine (27.7 mL, 343 mmol), acetic anhydride (15.5 mL, 164 mmol), and 4-(N,N-dimethylamino)pyridine (0.55 g, 4.5 mmol). After stirring for twelve hours at room temperature, the reaction was quenched with a saturated, aqueous, ammonium chloride solution (250 mL). The separated aqueous phase was extracted with dichloromethane (3×75 mL), and the combined organic phase washed sequentially with a 0.1 N hydrochloric acid solution (2×50 mL), and a saturated, aqueous, sodium bicarbonate solution (50 mL). The organic phase was dried over anhydrous sodium sulfate, filtered and concentrated in vacuo to a second brown oil. After toluene was added and removed in vacuo (3×), the resulting brown solid was crystallized from ethyl acetate-hexane to yield a first crop of the desired product (19.0 g). The mother liquor was concentrated and purified by flash column chromatography on silica gel, eluting with ethyl acetate-hexane (1:4), to obtain a second crop (5.0 g). The combined crops afforded the title compound as a homogeneous, colorless, crystalline, solid (24.0 g, 70%). m.p. 123-124° C.;

MS [(+ESI), m/z]: 230 [M+H]⁺;

¹H NMR (500 MHz, DMSO-d₆) δ: 9.24 (s, 1H), 7.44-7.23 (m, 8H), 1.87 (s, 3H);

Anal. calcd for C₁₄H₁₂FNO: C, 73.35; H, 5.28; N, 6.11. Found: C, 73.09; H, 5.20; N, 5.89.

### Step 2: 8-Fluoro-6-methylphenanthridine

The N-(4′-fluorobiphenyl-2-yl)acetamide (18.5 g, 80.7 mmol) was mixed with polyphosphoric acid (250 g) and heated at 120° C. with vigorous stirring for 48 hours. The hot reaction mixture was poured onto ice and stirred vigorously until homogeneous. Ammonium hydroxide (28-30%, aqueous) was added until the pH was greater than eight. A white precipitate was filtered, dissolved in ethyl acetate (250 mL), and re-filtered. The combined filtrate was washed with a saturated, aqueous, sodium chloride solution, dried over anhydrous sodium sulfate, filtered, and evaporated in vacuo to a brown solid. The brown solid was purified by crystallization from a mixture of ethyl acetate-hexane to yield the title compound as a white, crystalline solid (15.9 g, 94%), m.p. 92-93° C.;

MS [(+ESI), m/z]: 212 [M+H]⁺;

¹H NMR (500 MHz, CDCl₃) δ: 8.63 (dd, J=9.0, 5.4 Hz, 1H), 8.49 (dd, J=8.2, 1.0 Hz, 1H), 8.10 (dd, J=8.1, 1.1 Hz, 1H), 7.84 (dd, J=9.6, 2.6 Hz, 1H), 7.71 (m, 1H), 7.65-7.57 (m, 2H), 3.01 (s, 3H);

¹H NMR (400 MHz, DMSO-d₆) δ: 8.89 (dd, J=9.1, 5.6 Hz, 1H), 8.70 (dd, J=8.1, 1.3 Hz, 1H), 8.05 (dd, J=10.1, 2.5 Hz, 1H), 7.97 (dd, J=8.1, 1.3 Hz, 1H), 7.80 (m, 1H), 7.70 (m, 1H), 7.63 (m, 1H), 3.01 (s, 3H);

Anal. calcd for C₁₄H₁₀FN.0.10 H₂O: C, 78.93; H, 4.83; N, 6.57. Found: C, 78.90; H, 4.57; N, 6.58.

### Step 3: 4-(Chlorosulfonyl)phenyl ethyl carbonate

A solution of sodium 4-hydroxybenzenesulfonate dihydrate (50.0 g, 215 mmol) in 1.25 N aqueous sodium hydroxide (170 mL, 213 mmol) was treated drop-wise with ethyl chloroformate (20.6 mL, 215 mmol). The reaction mixture was stirred for twelve hours at room temperature. After cooling the mixture to 0° C., a white precipitate, which formed under the reaction conditions, was filtered. The solid was dried in vacuo at 70° C. The white solid (40.0 g) was suspended in toluene (350 mL) and treated with N,N-dimethylformamide (6.0 mL) and thionyl chloride (22.0 mL, 298 mmol), and the resulting mixture was heated at 100° C. for twelve hours. After cooling to room temperature, the reaction mixture was filtered through diatomaceous earth. The filtrate was concentrated in vacuo, and the resulting oil solidified upon standing. The solidified oil was dissolved in ethyl acetate-hexane (1:4), filtered through a short column of silica gel, and the solvent removed in vacuo to yield the sulfonyl chloride as a white solid (34.8 g, 61%), m.p. 74-76° C.;

¹H NMR (400 MHz, DMSO-d₆) δ: 7.60 (d, J=8.7 Hz, 2H), 7.14 (d, J=8.8 Hz, 2H), 4.23 (q, J=7.1 Hz, 2H), 1.26 (t, J=7.1 Hz, 3H).

### Step 4: Ethyl 4-[(8-fluoro-6-methylphenanthridin-5(6H)-yl)sulfonyl]phenyl carbonate

A stirred solution of 8-fluoro-6-methylphenanthridine (8.00 g, 37.9 mmol) in tetrahydrofuran (152 mL) was treated with freshly crushed sodium borohydride (7.16 g, 189 mmol). Trifluoroacetic acid (11.7 mL, 152 mmol) was added drop-wise at a rate suitable to control gas evolution and exothermic reaction conditions. After the trifluoroacetic acid addition was completed, the heterogeneous reaction mixture was stirred until the reaction returned to room temperature; then was re-heated to reflux for 14 hours. After cooling to room temperature, a saturated, aqueous, sodium bicarbonate solution (250 mL) was slowly added. The mixture was filtered through a plug of glass wool, and extracted with diethyl ether (4×75 mL). The combined organic extract was dried over anhydrous sodium sulfate, filtered, and concentrated in vacuo to afford the dihydrophenanthridine as a light-brown paste. A solution of the crude dihydrophenanthridine in dichloromethane (38 mL) was treated with triethylamine (31.7 mL, 227 mmol) and 4-(chlorosulfonyl)phenyl ethyl carbonate (12.0 g, 45.3 mmol), and stirred at room temperature for 14 hours. The reaction was quenched with a 0.1 N sodium hydroxide solution (150 mL) and extracted with dichloromethane (6×50 mL). The combined organic extract was washed with a 2 N hydrochloric acid solution (2×40 mL), dried over anhydrous sodium sulfate, filtered, and concentrated in vacuo to a viscous, brown oil. The brown oil was triturated with hexane (25 mL) to afford a light-brown solid. The light-brown solid was purified by crystallization from a mixture of ethyl acetate-hexane to yield a first crop of the desired product. The mother liquor was concentrated in vacuo, and purified by filtration through a plug of silica gel, eluting with ethyl acetate-hexane (1:4), to obtain a second crop. The combined crops afforded the title compound as a white, crystalline solid (15.2 g, 91%), m.p. 136-138° C.;

MS [(+ESI), m/z]: 442 [M+H]⁺;

¹H NMR (500 MHz, DMSO-d₆) δ: 7.77 (d, J=7.6 Hz, 1H), 7.63 (d, J=7.8 Hz, 1H), 7.48-7.39 (m, 3H), 7.19 (dd, J=9.0, 2.6 Hz, 1H), 7.09 (d, J=8.7 Hz, 2H), 6.98 (d, J=8.7 Hz, 2H), 6.93 (td, J=8.7, 2.6 Hz, 1H), 5.48 (q, J=7.0 Hz, 1H), 4.21 (q, J=7.1 Hz, 2H), 1.25 (t, J=7.1 Hz, 3H), 1.15 (t, J=7.0 Hz, 3H);

Anal. calcd for C₂₃H₂₀FNO₅S: C, 62.57; H, 4.57; N, 3.17. Found: C, 62.51; H, 4.47; N, 2.96.

### Step 5: 4-[(8-Fluoro-6-methylphenanthridin-5(6H)-yl)sulfonyl]phenol

A solution of ethyl 4-[(8-fluoro-6-methylphenanthridin-5(6H)-yl)sulfonyl]phenyl carbonate (0.45 g, 1.02 mmol) in methanol (5.0 mL) was treated with a 1 N sodium hydroxide (5.1 mL) solution, and heated at 75° C. for 14 hours. After cooling to room temperature, the methanol was evaporated in vacuo. The resulting aqueous mixture was acidified with a 1 N hydrochloric acid solution, diluted with a saturated, aqueous, sodium chloride solution (100 mL), and extracted with dichloromethane (5×15 mL). The combined organic phase was dried over anhydrous sodium sulfate, filtered, and concentrated in vacuo to a white solid. The solid was purified by filtration through a short column of silica gel, eluting with ethyl acetate, to yield the title compound as a homogeneous, white, crystalline, solid (0.34 g, 89%),

m.p. 188° C.;

MS [(−ESI), m/z]: 368 [M−H]⁻;

¹H NMR (500 MHz, DMSO-d₆) δ: 10.24 (br s, 1H), 7.76 (dd, J=7.6 Hz, 1.5, 1H), 7.60 (dd, J=7.8, 1.4 Hz, 1H), 7.52 (dd, J=8.7, 5.0 Hz, 1H), 7.41 (m, 1H), 7.37 (m, 1H), 7.17 (dd, J=9.2, 2.7 Hz, 1H), 6.96 (td, J=8.7, 2.7 Hz, 1H), 6.86 (d, J=8.9 Hz, 2H), 6.38 (d, J=8.9 Hz, 2H), 5.41 (q, J=7.0 Hz, 1H), 1.13 (d, J=7.0 Hz, 3H);

Anal. calcd for C₂₀H₁₆FNO₃S: C, 65.03; H, 4.37; N, 3.79. Found: C, 64.77; H, 4.31; N, 3.76.

### Example 4

### (S)-3-[4-(3,5-dimethoxyphenyl)piperazin-1-yl]-2-[(S)-(2-methoxyphenyl)(1-naphthyl)methyl]-2-methyl-3-oxopropanenitrile

A solution of (S,S)2-cyano-3-(2-methoxy-phenyl)-2-methyl-3-naphthalen-1-yl-propionic acid (0.45g, 1.30 mmol) in THF (150 mL) is treated with DMF (2 drops). Oxalyl chloride (0.16mL, 1.84 mmol) is added dropwise in order to control gas evolution; when the gas evolution stopped the solution is heated to reflux for 5 minutes. The solution is cooled, the THF is evaporated in vacuo and the solid is dissolved in dry toluene (15 mL) and evaporated to a solid. This procedure is repeated twice. The acid chloride is dissolved in dichloromethane (10 mL) this is added to a solution of 1-(3,5-dimethoxy-phenyl)-piperazine (305mg, 1.36 mmol) and a crystal of DMAP in dichloromethane (15 mL). This is followed by the dropwise addition of TEA (0.6 mL, 4.27 mmol). The reaction is stirred overnight. The reaction mixture is diluted with dichloromethane (50 mL), washed with aqueous HCl (10 mL 0.5N) then saturated NaHCO₃ (10 mL) and brine (10 mL). The sample is dried over NaSO₄, filtered and concentrated in vacuo. Chromatography on silica gel using 30% ethyl acetate/hexanes provided 510 mg the title compound as a white solid. Recrystallization from ethyl acetate/hexanes yielded colorless needles.

mp 186-188° C.;

[α]_{D}²⁵=−153.91° (1%, CHCl₃);

¹H NMR 500 MHz (DMSO-D6): δ 7.96 (d,1H, J=7.33 Hz), 7,86 (m, 2H), 7.78 (d,1H, J=8.24 Hz), 7.53 (t, 1H, J=7.94 Hz), 7.22 (t, 1H, J=7.48 Hz), 7.12 (m, 2H), 6.79 (t,1H, J=7.49 Hz), 6.01 (m, 4H), 4.01 (s,3H), 3.68 (s,6H), 3.05 (brs,4H), 1.63 (s,3H)

(ESI) m/z 550 ([M+H]+);

Anal. calcd for C₃₄H₃₅N₃O₄: C, 74.29; H, 6.42; N, 7.64; Found: C, 74.10; H, 6.35; N, 7.87.

### Example 5

### (S,S)-3-(2-methoxyphenyl)-2-methyl-3-(1-naphthyl)-2-({4-[3-(trifluoromethyl)phenyl]piperidin-1-yl}carbonyl)propanenitrile

The title compound was prepared in 75% yield according to Example 4 using 4-(3-trifluoromethyl-phenyl)-piperidine. Recrystallization from ethyl acetate/hexane yielded white crystals.

mp 123-127° C.;

[α]_{D}²⁵=−144.40 (1%, CHCl₃);

¹H NMR 500 MHz (DMSO-D6): δ 8.02 (d,1H, J=7.03 Hz), 7.88 (m,2H), 7.82 (d,1H, J=8.25 Hz), 7.56 (t,1H, J=7.79 Hz),7.51 (brm,2H), 7.42 (d,1H, J=7.48 Hz), 7.42 (quin,1H, J=3.51 Hz), 7.23 (t,1H, J=7.03 Hz), 7.14 (m,2H), 6.80 (t,1H, J=7.48 Hz), 6.02 (s,1H),4.37 (brm, 2H), 4.01 (s, 3H), 2.88 (brm, 2H), 1.66 (s, 3H)

MS (ESI) m/z 557 ([M+H]+);

Anal. calcd for C₃₄H₃₁F₃N₂O₂: C, 73.35; H, 5.61; N, 5.03; Found: C, 73.99; H, 6.00; N, 4.72

### Example 6

### Animal Procedures

Methods

All animal procedures were performed under strict compliance to IACUC guidelines. Four to six-week old female apolipoprotein E-deficient C57/B1J (apo E KO) mice were ovariectomized by the vendor (Taconic) and delivered to Wyeth within 2 days of surgery. The animals were housed in shoe-box cages and were allowed ad lib food and water. After 3 days acclimation, the animals were randomized by weight into 8 groups (N=12 mice per group). The animals were dosed in the feed with the indicated compounds during the entire course of the study. Dosing at the indicated levels was achieved by blending the compounds with a sucrose vehicle (30 g per 2 kg feed) prior to mixing into the feed. Ethinyl estradiol was used as a positive reference compound and was dosed at 12 μg/kg/d. Other groups of animals were dosed with the compounds of Example 3 or Example 5 at either 10 or 25 mg/kg/d. Control animals received vehicle (sucrose) alone in the feed. The quantity of compound added to the feed was based on the food consumption rate and the body weights of the animals, which was determined every week. The feeders employed prevented any spillage, so that the food consumption rate was accurate. The animals were fed a Casein-based rodent chow meal (Purina #5K96M) for the first week of the study. The animals were then challenged with a casein-based high fat (HF) diet for week 2 to week 7 of the study. The HF diet (#591-A) was prepared by Purina and contained 1.5% cholesterol, 20% fat as cocoa butter and contained no cholic acid. At the end of the study period, the animals were euthanized and plasma samples obtained. The aortas were perfused via the left ventricle first with buffered saline and then with neutral buffered 10% formalin solution.

Lipoprotein Determinations—Total cholesterol and triglycerides were determined using enzymatic methods with commercially available kits from Boehringer Mannheim and Wako Biochemicals, respectively, and analyzed with the Boehringer Mannheim Hitachii 911 Analyzer. Separation and quantitation of plasma lipoproteins were performed using FPLC size fractionation. Briefly, 50-100 μl serum was filtered and injected into Superose 12 and Superose 6 columns connected in series and eluted at a constant flow rate using 0.15 M NaCl. Areas of each curve representing VLDL, LDL and HDL were integrated using Waters Millennium™ software, and each lipoprotein fraction was quantified by multiplying the Total Cholesterol value by the relative percent area of each respective chromatogram peak.

Aortic Atherosclerosis Quantification-The mice were euthanized by anesthesia overdose and the vascular tree was perfused first with 2 ml phosphate buffered saline (pH 7.4), then with 2 ml formalin. For quantification of aortic lesions, aortas were carefully isolated and placed in formalin fixative for 48-72 hours before handling. Atherosclerotic lesions were identified by Oil Red O staining. The vessels were briefly destained then imaged using a Nikon SMZ800 microscope fitted with a Spot Diagnostic Instruments RT color digital camera using Image-Pro Plus Version 4.5 (Media Cybernetics) as the image capturing software. The lesions were quantified en face after staining along the aortic arch using the Image-Pro Plus, Media Cybernetics software. Automated lesion assessment was performed on the vessels in a blinded fashion using the threshold function of the program, specifically on the region contained within the aortic arch from the proximal edge of the brachiocephalic trunk to the distal edge of the left subclavian artery. Aortic atherosclerosis data were expressed as percent lesion involvement strictly within this defined luminal area.

Results

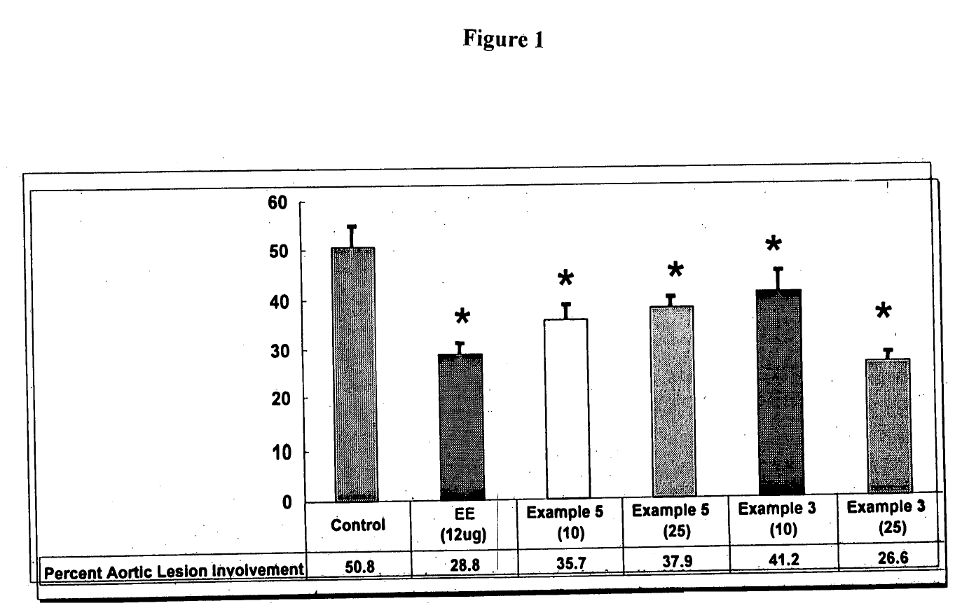

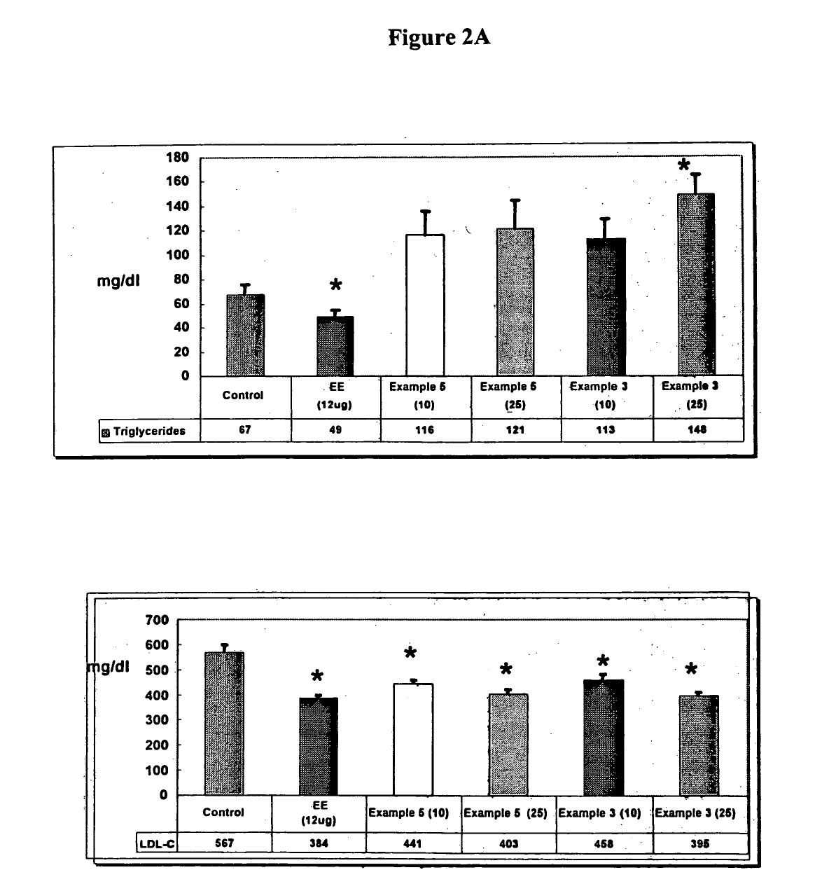

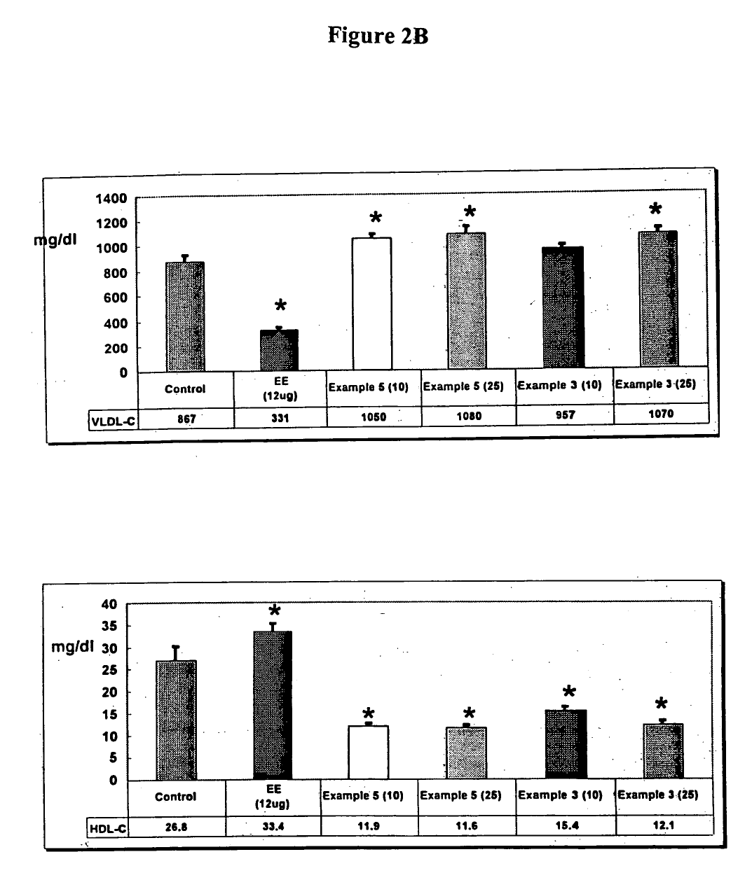

Ovariectomized apoE KO mice fed an atherogenic diet for 6 weeks were treated orally with 17α-ethinyl-17β-estradiol (EE) or two different NF-kB selective ER ligands, the compounds of example 3 and 5. Dosing with EE, the compound of example 3 or the compound of example 5 (N=12) was associated with a significant decrease in the extent of aortic fatty streak involvement. The compound of example 3 at 25 mg/kg/day provided a protection against atherosclerotic lesion development that was similar to that of EE (FIG. 1). This beneficial effect on aortic atherosclerosis in the EE-treated group was associated with a significant reduction of serum total cholesterol, VLDL-C and an elevation of HDL-C (FIG. 2). In contrast, although protective against atherosclerosis, dosing with the compounds of examples 3 or 5 were each associated with an increase in VLDL-C, no effect on total cholesterol and significantly lower serum HDL-C. Thus, the protective ability of these compounds appears to be independent of changes in lipoprotein metabolism in this model.

These results indicate that ER ligands that selectively interfere with NF-κB activity may have utility in treating atherosclerosis independent of lipid lowering. It is believed that the reduction in lesion progression observed here may occur through inhibition of the recruitment of inflammatory cells by directly interfering with inflammatory gene expression.

All patents, publications, and other documents cited herein are hereby incorporated by reference in their entirety.

1. A method of treating atherosclerosis comprising the steps:
identifying a person in need of treatment for atherosclerosis; and
administering to said person a therapeutically effective amount of a ligand which modulates NF-kB transcription factor by interaction with estrogen receptor ER-α, estrogen receptor ER-β, or both ER-α and ER-β estrogen receptors with a substantial absence of creatine kinase stimulation.

2. The method of claim 1 wherein said administration is with a substantial absence of uterotropic activity.

3. The method of claim 2 wherein the ligand interacts with ER-β.

4. The method of claim 2 wherein the ligand interacts with ER-α.

5. The method of claim 2 wherein the ligand interacts with both ER-α and ER-β.
